(19) United States
(12) Patent Application Publication
(10) Pub. No.: US 2012/0309018 A1
(43) Pub. Date: Dec. 6, 2012

(54) CANCER DETECTION MARKERS

(75) Inventors: Mark Skolnick, Salt Lake City, UT (US); Jerry Lanchbury, Salt Lake City, UT (US); Kirsten Timms, Salt Lake City, UT (US); Chris Neff, Salt Lake City, UT (US); Ryan Hoff, Salt Lake City, UT (US); Hubert Wang, Salt Lake City, UT (US); Susanne Wagner, Salt Lake City, UT (US); Zaina Sangale, Salt Lake City, UT (US)

(73) Assignee: Myriad Genetics, Incorporated, Salt Lake City, UT (US)

(21) Appl. No.: 13/154,143

(22) Filed: Jun. 6, 2011

(51) Int. Cl. G01N 33/574; G01N 33/577

(52) U.S. Cl. 435/7.4; 436/501; 435/7.92

(57) ABSTRACT

Methods and compositions involving molecular markers for the detection and characterization of cancer in a patient are provided.

### CROSS-REFERENCE TO RELATED APPLICATIONS

This application is the U.S. national phase of International Application No. PCT/US2009/067029 filed on Dec. 7, 2009, which designated the U.S. and claims priority to U.S. Provisional Patent Application Ser. No. 61/175,954 filed May 6, 2009, U.S. Provisional Patent Application Ser. No. 61/174,600 filed May 1, 2009, U.S. Provisional Patent Application Ser. No. 61/158,975 filed Mar. 10, 2009, U.S. Provisional Patent Application Ser. No. 61/120,259 filed Dec. 5, 2008, the entire contents of each of which are hereby incorporated by reference.

### FIELD OF THE INVENTION

The invention generally relates to a molecular classification of disease and particularly to molecular markers for cancer and methods of use thereof.

### BACKGROUND OF THE INVENTION

Cancer is a major health challenge. Nearly 560,000 people die from cancer annually in the United States alone, representing almost 23% of all deaths. See American Cancer Society, Cancer Facts & Figures 2008, 1-2 (2008). Despite recent advances in molecular and imaging diagnostics, one of the most vexing aspects of cancer remains early detection. In fact, for certain types of cancer—e.g., pancreatic adenocarcinoma—detection often occurs so late as to practically preclude any good prognosis. Thus there is an urgent need for sensitive methods of detecting cancer.

### SUMMARY OF THE INVENTION

Small extracellular vesicles (e.g., exosomes), or the markers associated with them, are often found in altered status in the samples of patients having certain diseases or disease-related pathologies as compared to samples from healthy individuals. This is especially true of epithelial cancers (e.g., those of the lung, colon, breast, prostate, ovaries, endometrium, etc.), diabetic nephropathy, etc.

Thus one aspect of the invention provides a method for detecting cancer in a patient comprising determining the status of exosomes and/or an exosome-associated marker in a sample obtained from a patient, wherein an abnormal exosome (and/or exosome-associated marker) status in the sample indicates the presence of cancer. In some embodiments status is determined by determining the level of exosomes or an exosome-associated marker in the sample. In some embodiments determining the level of exosomes in the sample comprises measuring the aggregate level of all exosomes in a sample. In some embodiments status is determined by measuring the level of exosomes bearing a particular marker. This may comprise determining the status (e.g., the level) of an exosome-associated marker. It may also comprise isolating exosomes from the sample and determining the status (e.g., the level) of an exosome-associated marker. In some embodiments the exosome-associated marker is a cancer-marker, a cancer-type marker, and/or a tissue-type marker. Examples of exosome-associated markers include those listed in Table 1.

Some markers are correlated with cancer of a specific type (e.g., cell type, tissue type, organ, clinical subtype, etc.). Thus another aspect of the invention provides a method of diagnosing a cancer in a patient comprising determining the status of an exosome-associated cancer-type marker in a sample obtained from the patient, wherein an abnormal status of the marker indicates the presence of a specific cancer type, or cancer in a specific tissue or organ, in the patient. In some embodiments the exosome-associated marker is not necessarily correlated with cancer, but with a particular tissue type or organ. An example of such an analysis is given in FIG. 2. In some embodiments exosomes are isolated and the exosome-associated marker is then analyzed. Thus one embodiment of the invention provides a method of diagnosing a cancer in a patient comprising (1) isolating exosomes from a sample obtained from a patient and (2) determining the status of a cancer-type marker associated with the exosomes isolated in (1); wherein an abnormal status of the marker in (2) indicates the presence of indicates the presence of a specific cancer type, or cancer in a specific tissue or organ, in the patient. An example of such an analysis is given in FIG. 2. Examples of cancer-type markers include some of those listed in Table 1 (e.g., EGFRvIII, ErbB2).

Another aspect of the invention provides a method of screening for cancer in a patient comprising identifying a patient at risk of having cancer or in need of screening and determining the status of exosomes and/or an exosome-associated marker in a sample obtained from the patient, wherein an abnormal status of exosomes and/or the exosome-associated marker in the sample indicates the presence of cancer. In some embodiments the patient is at risk for developing a specific cancer type and the abnormal status of exosomes and/or the exosome-associated marker indicates the presence of this specific cancer type.

Yet another aspect of the invention provides a method of detecting recurrence in a cancer patient comprising determining the status of exosomes and/or an exosome-associated marker in a sample obtained from a patient, wherein an abnormal status of exosomes and/or the exosome-associated marker in the sample indicates recurrence.

Still another aspect of the invention provides a diagnostic method comprising identifying a patient who is a candidate for biopsy and determining the status of exosomes and/or an exosome-associated marker in a sample obtained from the patient, wherein an abnormal status of exosomes and/or the exosome-associated marker in the sample indicates a biopsy is desirable. In some embodiments no abnormal status of exosomes and/or the exosome-associated marker indicates no biopsy is necessary.

One aspect of the invention provides a method of detecting a specific disease other than cancer (e.g., rheumatoid arthritis, diabetic nephropathy) comprising determining the status of exosomes and/or an exosome-associated marker in a sample obtained from a patient, wherein an abnormal status of exosomes and/or the exosome-associated marker indicates the patient has the disease.

Various techniques can be used to determine the status of exosomes. Examples of such techniques include, but are not limited to: flow cytometry; enzyme-linked immunosorbent assay (ELISA) and its numerous variations (e.g., electrochemiluminescence (ECL)); immunohistochemistry (IHC); etc. or any combination of these. Exosomes can be isolated by various techniques including, but not limited to: centrifugation (e.g., ultracentrifugation); physical filtration; affinity chromatography; contacting a sample with a solid surface (e.g., plate, well, bead, etc.) containing an antibody against an exosome-associated marker in order to attach any exosomes to the surface; etc. or any combination of these.

Another aspect of the invention provides exosome blocks comprising fixed (e.g., formalin-fixed) exosomes embedded in some medium (e.g., paraffin). Such exosome blocks allow for long-term storage of exosomes and also allow for analysis of the interior of exosomes.

These aspects of the invention provide a relatively quick and inexpensive screen for the presence of cancer. This can be used as a general population screen (e.g., yearly blood screen) whose ease, non-invasiveness, patient inclusivity, and sensitivity may provide vast improvements over existing general screens such as mammograms, colonoscopies, etc.

Unless otherwise defined, all technical and scientific terms used herein have the same meaning as commonly understood by one of ordinary skill in the art to which this invention pertains. Although methods and materials similar or equivalent to those described herein can be used in the practice or testing of the present invention, suitable methods and materials are described below. In case of conflict, the present specification, including definitions, will control. In addition, the materials, methods, and examples are illustrative only and not intended to be limiting.

Other features and advantages of the invention will be apparent from the following detailed description, and from the claims.

### BRIEF DESCRIPTION OF THE DRAWINGS

FIG. 1 illustrates a method, according to the invention, for detecting cancer generally and also determining the specific cancer type;

FIG. 2 illustrates one embodiment of the invention using various biomarkers to determine which specific cancer is present in a patient.

FIGS. 3-7 illustrate differentiating cancer patients from cancer-free controls by determining exosome status using several different techniques.

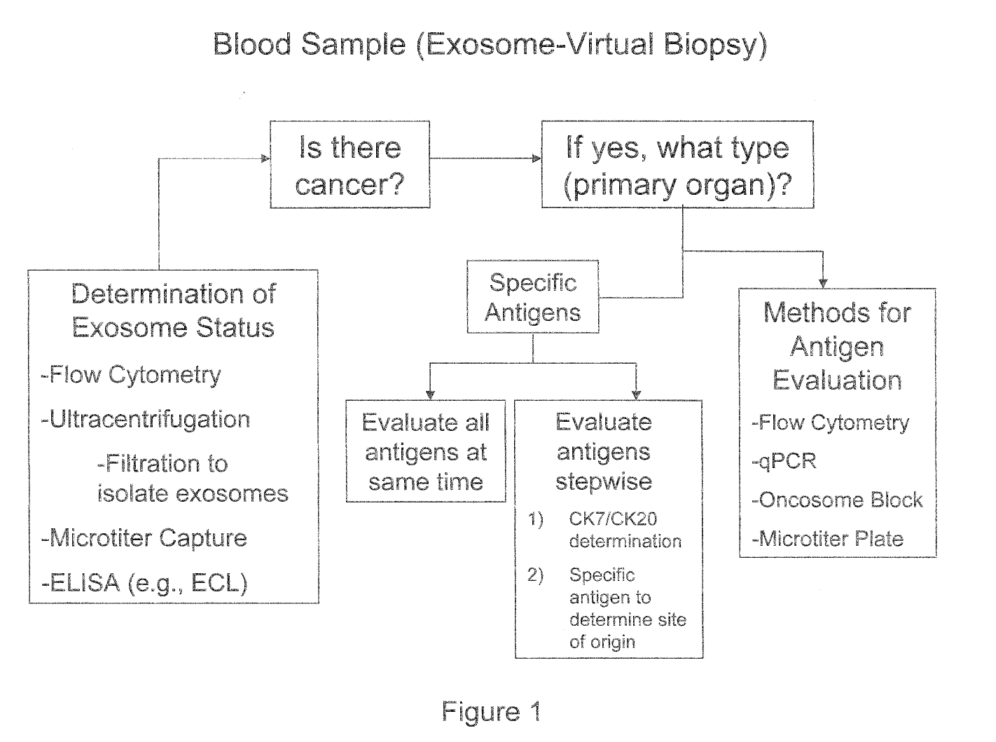

### DETAILED DESCRIPTION OF THE INVENTION

In addition to cells, blood contains various additional circulating elements. These include, but are not limited to, cellular debris (e.g., membrane fragments), free protein, protein aggregates, and small extracellular vesicles. It has been discovered that markers carried by these elements can be analyzed in order to detect cancer in patient samples. More specifically, it has been discovered that capturing these items with a capture reagent and then detecting them with a detection reagent can separate cancer patients from controls. For example, small extracellular vesicles called exosomes have been isolated from patient samples and their status analyzed by way of exosome-associated markers so as to differentiate healthy from diseased individuals. In another example cancer has been detected by subjecting plasma samples to various antibodies and integrins to capture circulating elements and analyzing these elements by way of detection antibodies.

Cells form several different small vesicles within their interior that perform various functions. For example, lysosomes digest excess or worn-out organelles, food particles, and engulfed viruses or bacteria. Some vesicles are trafficked from the cell interior to outside the cell. These include exosomes, which are found in the cell within the multi-vesicular body (MVB) and released into the extracellular matrix and into various bodily fluids such as blood, serum, urine, etc. These vesicles often bear, either inside or on their surface, various biological molecules such as protein, RNA, DNA, etc. Exosomes are secreted by a variety of cells, including those involved in immune response (T cells, B cells, dendritic cells, macrophages) and epithelial cells.

Exosomes have been determined to be useful markers in detecting and characterizing cancer. For example, the status of exosomes in a sample has been found to correlate strongly with the presence of cancer in the patient from whom the sample was obtained. More specifically, elevated exosomes and/or exosome-associated markers in a patient sample indicate the patient has cancer. See Examples below. Further, exosome-associated markers can help in characterizing the patient's specific cancer (e.g., cell type, tissue type, organ, clinical subtype, etc.).

Thus one aspect of the invention provides a method for determining whether an individual has cancer comprising determining the status of exosomes and/or an exosome-associated marker in a sample obtained from the individual, wherein an abnormal exosome (and/or exosome-associated marker) status indicates an increased likelihood of cancer. As used herein, the “status” of a biomolecular marker (e.g., exosomes, exosome-associated markers, etc.) refers to the presence, absence, or extent/level of the marker or some physical, chemical, or genetic characteristic of the marker or its expression product(s). Such characteristics include, but are not limited to, expression level, activity level, structure (e.g., sequence information, including mutations), copy number, post-translational modification for proteins (e.g., glycosylation), etc. These may be assayed directly (e.g., by assaying the expression level of a particular gene) or determined indirectly (e.g., assaying the level of marker whose expression level is correlated to the expression level of the gene of interest).

In some embodiments determining the status of exosomes and/or an exosome-associated marker comprises determining their level in a sample. As used herein in the context of exosomes, markers, etc., the “level” of something in a sample has its conventional meaning in the art. Determining a “level” therefore includes quantitative determinations—e.g., mg/mL, fold change, etc. Determining a “level” herein also includes more qualitative determinations—e.g., determining the presence or absence of a marker or determining whether the level of the marker is “high,” “low” or even “present” relative to some index value.

“Abnormal status” means a marker's status in a particular sample differs from the reference status for that marker (e.g., in healthy samples or average diseased samples). Examples include mutated (when the sequence or structure of the marker is analyzed), elevated, decreased, present, absent, etc. For example, determining the status of an extracellular protein marker will often include determining its level in a sample (e.g., on the surface of exosomes). An abnormal status could be either lower (including undetectable) or higher levels (including anything non-zero) compared to the index value in exosome samples from healthy patients. Another example of abnormal status includes a sequence (i.e., structural) variation in a protein or gene, such as EGFRvIII, or in an mRNA, such as KRAS. In this context, a patient has an “increased likelihood of cancer” if the status of the relevant marker in the patient's sample is correlated with cancer. Examples include mutations in particular genes correlated with cancer, a level of the marker that is closer to some cancer index value than to a normal index value, a level of the marker that exceeds some threshold value where exceeding that value is correlated with cancer, etc. Thus “increased likelihood of cancer” means a patient with an abnormal status for a marker has a higher likelihood of cancer than if the patient did not have an abnormal status.

An “elevated status” means that one or more of the above characteristics (e.g., expression) of the marker of interest is higher than normal levels. Generally this means an increase in the characteristic (e.g., expression) as compared to an index value. Conversely a “low status” means that one or more of the above characteristics (e.g., expression) is lower than normal levels. Generally this means a decrease in the characteristic (e.g., expression) as compared to an index value. In this context, a “negative status” generally means the characteristic is absent or undetectable (which would include expression, copy number, methylation, etc.).

In some embodiments, the level of a marker is determined within one or more samples as compared to some index value. Those skilled in the art will appreciate how to obtain and use an index value in the methods of the invention. The index value may represent the average (e.g., mean) level in a plurality of training patients (e.g., both diseased and healthy patients). For example, a “cancer index value” can be generated from a plurality of training patients characterized as having cancer. A “cancer-free index value” can be generated from a plurality of training patients defined as not having cancer. Thus, a cancer index value may represent the average level of a marker (e.g., exosomes, exosome-associated markers, etc.) in patients having cancer, whereas a cancer-free index value may represent the average level of the marker in patients not having cancer. Thus, when the level of the marker is more similar to the cancer index value than to the cancer-free index value, then it can be concluded that the patient has or is likely to have cancer. On the other hand, if the level of the marker is more similar to the cancer-free index value than to the cancer index value, then it can be concluded that the patient does not have, or has no increased likelihood of, cancer.

Alternatively index values may be determined thusly: In order to assign patients to risk groups, a threshold value may be set for the marker. The optimal threshold value is selected based on the receiver operating characteristic (ROC) curve, which plots sensitivity vs. (1—specificity). For each increment of the marker mean (e.g., exosome-associated marker expression), the sensitivity and specificity of the test is calculated using that value as a threshold. The actual threshold will be the value that optimizes these metrics according to the artisan's requirements (e.g., what degree of sensitivity or specificity is desired, etc.).

Determining the status of exosomes in a sample may also comprise assaying some marker whose status itself is correlated with exosome status. This marker will often be an exosome-associated marker. “Exosome-associated” marker means a biomolecule inside or on the surface of an exosome. For example, exosomes carry cellular biomolecules such as proteins and nucleic acids (e.g., mRNA, microRNA, etc.) within their lipid membrane. Since exosomes are formed from membranes of the cell, they also carry molecules (e.g., cell-surface molecules such as CD63 or EpCAM) on their surface. Additionally, since some biomolecules bind those on the surface of exosomes (e.g., antibodies binding an exosome-surface protein), these biomolecules are also indirectly “exosome-associated.” As discussed below, in some embodiments the invention need not be limited by the actual physical element that is measured in the claimed method as long as the status of an exosome-associated marker such as those listed in Table 1 is determined. Thus, the invention envisions detecting an exosome-associated marker even after such marker has been separated from the exosome with which it is sometimes associated.

Thus, in one aspect the invention provides a method of detecting cancer in a patient comprising determining the status of an exosome-associated marker (e.g., the markers listed in Table 1) in a sample obtained from the patient, wherein an abnormal status for such exosome-associated marker indicates an increased likelihood of cancer. Markers useful in this aspect include those listed in Table 1, EpCAM, MUC1, CDH1, CK7, PSA, etc.

It has been determined that the status of each exosome-associated marker listed in Table 1 (alone or in combination) is correlated with the presence of cancer. See Examples below.

TABLE 1

| Marker Name/Symbol | Entrez GeneId No. |
|---|---|
| ADAM10 | 102 |
| αVβ6 integrin | 3678, 3694 |
| Caveolin-1 | 857 |
| CD147 | 382 |
| CD36 | 948 |
| CD63 | 967 |
| CD81 | 975 |
| Claudin-3 | 1365 |
| Claudin-4 | 1364 |
| Desmocollin-1 | 1823 |
| EGFR | 1956 |
| EGFRvIII | 1956 |
| EMP-2 | 2013 |
| EpCAM | 4072 |
| ErbB2 | 2064 |
| GP1b | 2811 |
| HLA-DR | Various |
| Hsp70 | 3308 |
| Hsp90 | 3320 |
| MFG-E8 | 4240 |
| Rab13 | 5872 |
| Coagulation factor III | 2152 |

Thus in some embodiments the invention provides a method of detecting cancer comprising determining the status of at least one marker chosen from the group consisting of: ADAM10, aαVβ6 integrin, Caveolin-1, CD147, CD36, CD63, CD81, Claudin-3, Claudin-4, Desmocollin-1, EGFR, EGFRvIII, EMP-2, EpCAM, ErbB2, GP1b, HLA-DR, Hsp70, Hsp90, MFG-E8, Rab13, and Tissue Factor, wherein an abnormal status indicates an increased likelihood of cancer.

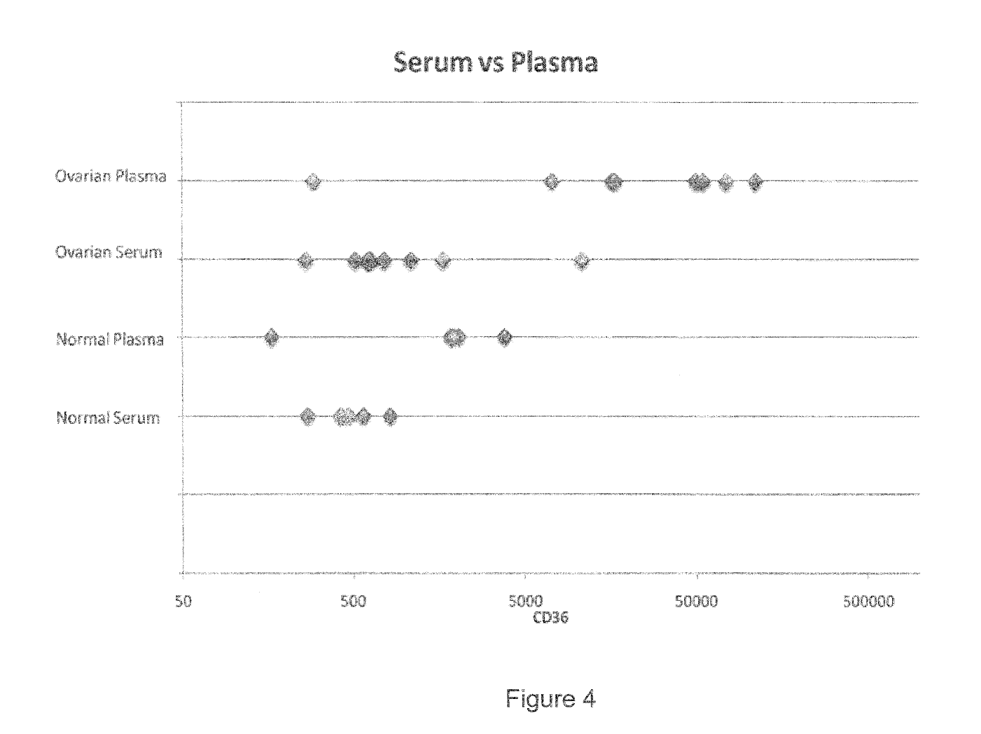

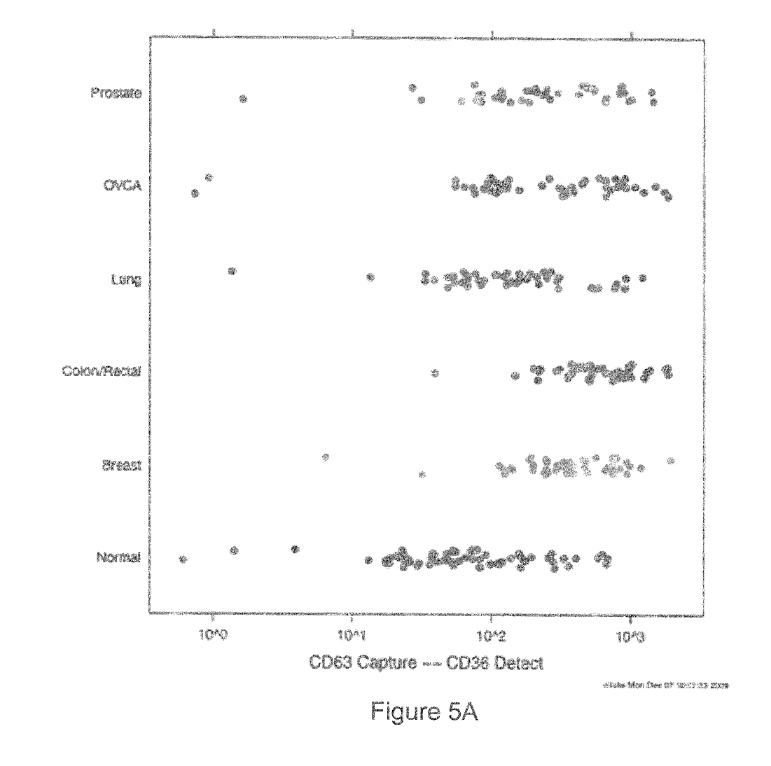

Patient blood samples are complex mixtures of circulating elements including, but not limited to, cellular debris (e.g., membrane fragments), free protein, protein aggregates, and small extracellular vesicles (e.g., exosomes). In some embodiments the patient blood sample is enriched for exosomes and/or exosome-associated markers. In other words the sample is enriched to remove cells and individual proteins. “Sample” as used herein refers to any biological specimen, including any tissue or fluid, that can be obtained from, or derived from a specimen obtained from, a human subject. Such samples include, healthy or tumor tissue, bodily fluids, waste matter (e.g., urine, stool), etc. In some embodiments the sample is blood or any substance derived therefrom—e.g., serum or plasma. The process of extracting plasma from blood, by removing the blood cells, helps to enrich for exosomes and/or exosomes-associated markers. Further purifying plasma to serum, by removing fibrinogen and other clotting factors, further enriches the sample. Additional enrichment/purification may be performed by various techniques discussed in more detail below including, but not limited to, ultracentrifugation, filtration, affinity chromatography, antibody capture, cell-sorting, etc. Though the methods of the invention can differentiate cancer patients from controls in both plasma and serum samples, it has been discovered that plasma samples yield better (i.e., clearer) differentiation. See FIG. 4.

Thus in one aspect the invention provides a method of detecting cancer comprising enriching a sample for exosomes and determining the status of the exosomes and/or an exosome-associated marker, wherein an abnormal status for exosomes and/or the exosome-associated marker indicates an increased likelihood of cancer. In some embodiments the exosome-associated marker is chosen from those listed in Table 1. In some embodiments the sample is blood plasma and the sample is enriched by a technique chosen from the group consisting of ultracentrifugation, filtration, affinity chromatography, antibody capture and cell-sorting. In some embodiments the sample is enriched using ultracentrifugation and the resulting exosomes (or other circulating elements) are coated onto a solid surface for subsequent detection (see Examples 2, 4 & 5). In some embodiments the sample is enriched using a capture antibody specific for one of the markers listed in Table 1 and the exosomes and/or exosome-associated markers are detected using an antibody specific for another marker listed in Table 1.

Though the inventors have devised assays aimed at measuring exosomes and exosome-associated markers from patient blood, plasma and serum, some aspects of the invention need not be limited to the particular circulating element that is actually measured. Specifically, in some examples the inventors have attempted to isolate/purify exosomes from blood (or plasma or serum) samples using antibody or integrin capture, ultracentrifugation and/or filtration, followed by detection (e.g., via ELISA, ECL, flow cytometry, Bradford protein measurement, etc.). See Examples 1, 2 & 5. In some cases, however, it is ultimately the choice of markers and/or assays that has enabled cancer detection regardless of whether exosomes themselves (or some other circulating element) have been directly measured. Thus, in some examples no significant attempt has been made at isolating or purifying exosomes other than using distinct capture and detection reagents (e.g., anti-CD63 antibody for capture and anti-Claudin-3 antibody for detection). See Example 4. Though not wishing to be bound by theory, it is thought that using distinct capture and detection reagents will not detect single circulating proteins and, in the case of plasma, will not detect whole cells. In the case of serum, clotting factors have further been eliminated. Such an assay should instead detect exosomes or small membrane fragments, protein complexes, etc. containing exosome-associated markers (e.g., those listed in Table 1).

Thus one aspect of the invention provides a method of detecting cancer comprising determining the status of at least two markers chosen from those listed in Table 1 in a patient sample, wherein an abnormal status for at least one of the markers indicates an increased likelihood of cancer. In some embodiments one of the markers is used to capture circulating elements from the sample while another of the markers is used to detect (e.g., quantify) these elements. In some embodiments the level of the markers is determined using an antibody specific for each marker. In some embodiments the status of the capture marker is its presence or absence, which is determined by attaching the capture marker to a solid surface via a capture reagent (e.g., antibody, nucleic acid probe, etc.), while the status of the other marker is its level as determined via a detection reagent (e.g., antibody, nucleic acid probe, etc.). The markers listed in Table 1 can be used in combination in this way, including but not limited to the combinations described in Example 4.

In some embodiments the exosome-associated marker is a cancer marker. As used herein, “cancer marker” means a biomarker whose status is correlated with the presence of cancer. There need not be a strong correlation between the biomarker and any specific cancer type. Examples include carcinoembryonic antigen (CEA) and epithelial membrane antigen (EMA). Another example includes EpCAM, which is hyperglycosylated in carcinoma tissue as compared to corresponding normal epithelial tissue. See, e.g., Munz et al., FRONT BIOSCI. (2008) 13:5195-5201. Thus some embodiments provide a method of detecting cancer comprising providing a sample obtained from a patient and determining the level of exosomes having a general cancer marker (e.g., hyperglycosylated EpCAM), wherein an increased level of these exosomes indicates an increased likelihood of cancer.

Some markers are correlated with cancer of a specific type (e.g., cell type, tissue type, organ, clinical subtype, etc.). For example, certain biomarkers can be used to differentiate exosomes derived from epithelial cells from other exosomes, e.g., immune cell-derived. One particularly useful marker in detecting, quantitating, collecting, and/or analyzing epithelial exosomes is EpCAM, a protein expressed on the surface of many epithelial cells. Other epithelial markers useful in the invention include, but are not limited to, CDH1 (cadherin 1, type 1, E-cadherin [epithelial]) and cytokeratin 7 (CK7 or KRT7). Elevated levels of epithelial exosomes are often found in individuals having an altered physiological condition. For example, pregnant women show higher levels of epithelial exosomes in their serum, as do diabetic patients suffering from nephropathy. Further, epithelial exosomes are more abundant in samples from cancer patients than in those from cancer-free controls.

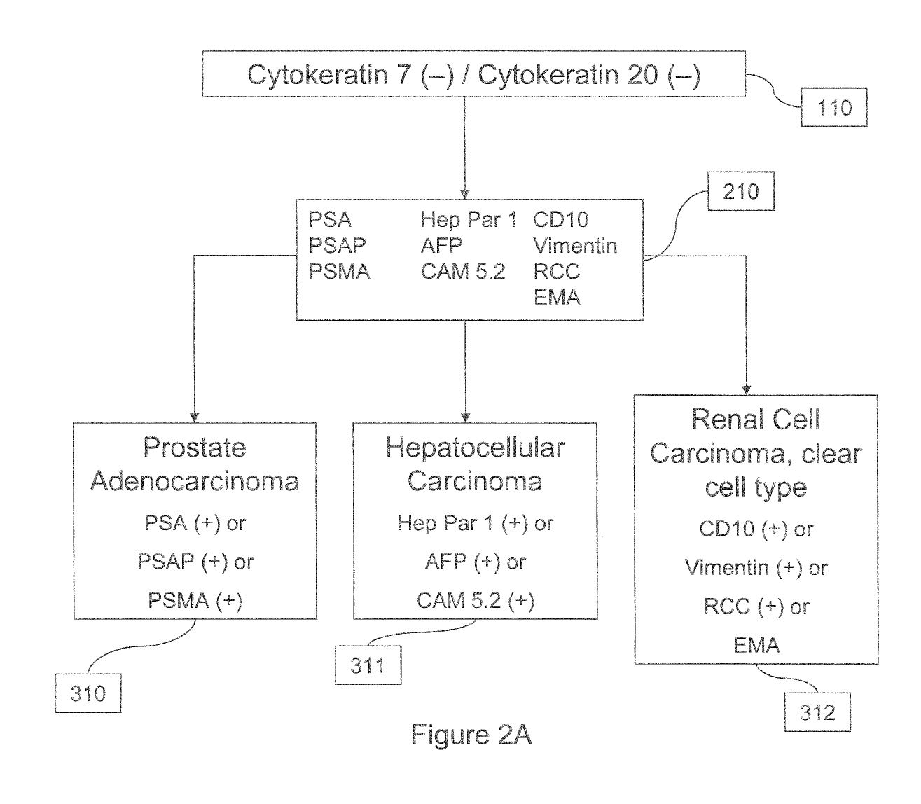

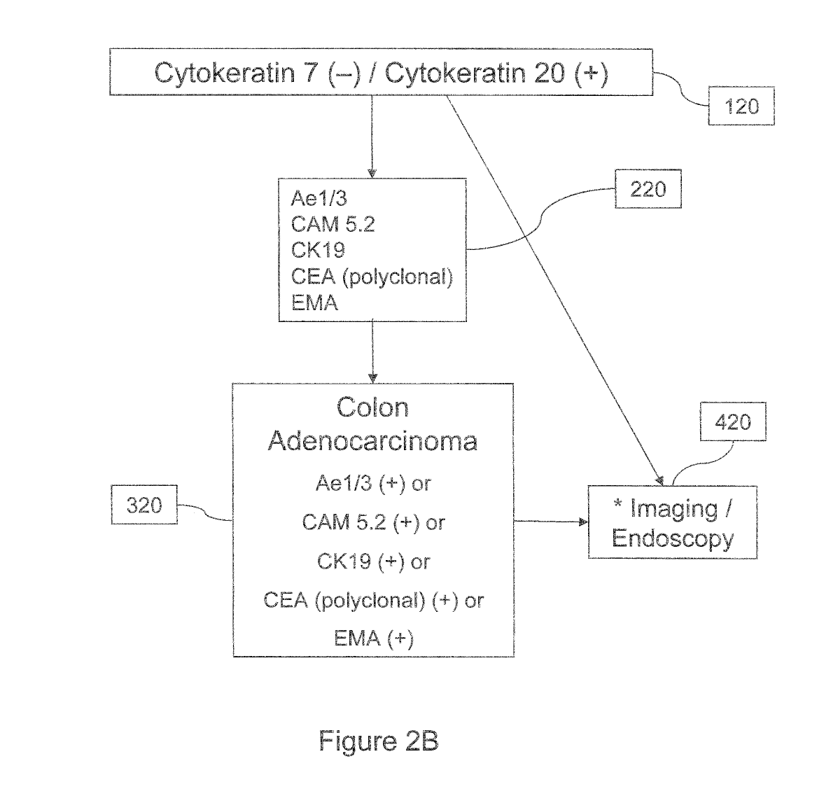

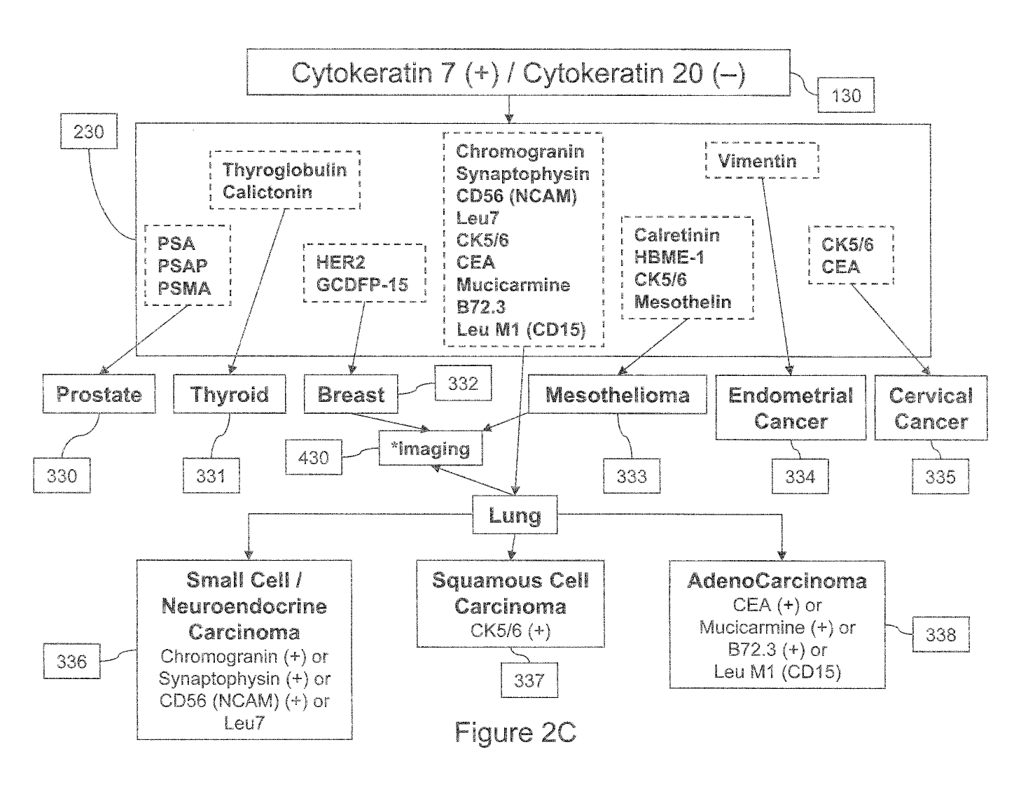

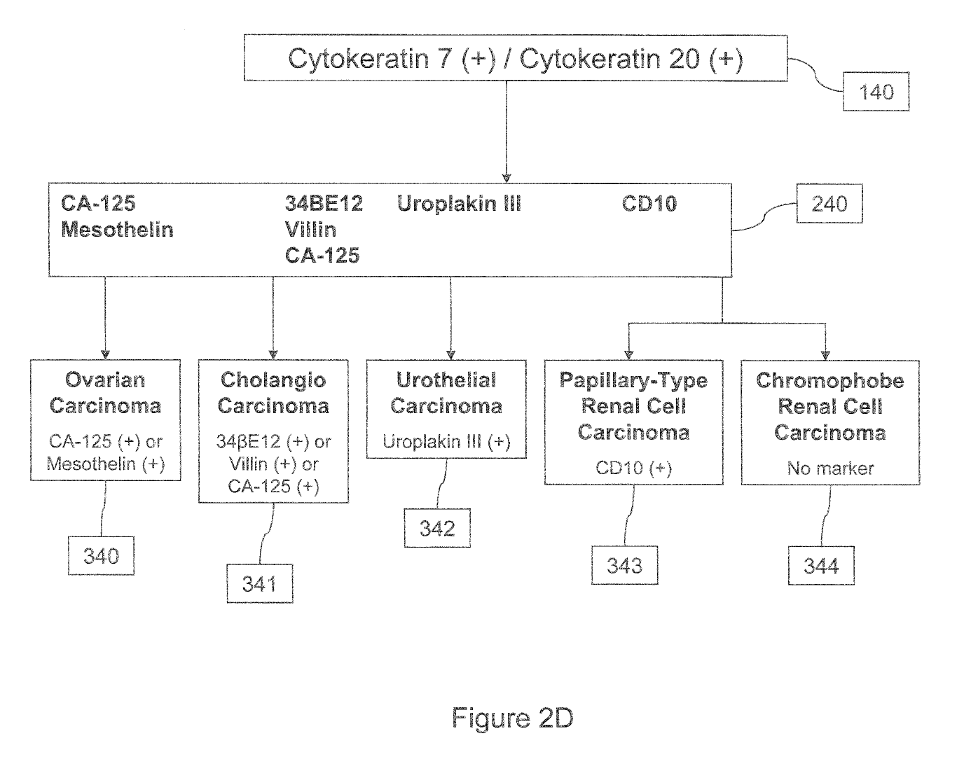

Thus another aspect of the invention provides a method of diagnosing a cancer in a patient comprising determining the status of an exosome-associated cancer-type marker in a sample obtained from the patient, wherein an abnormal status of the marker indicates the presence of a specific cancer type, or cancer in a specific tissue or organ, in the patient. In some embodiments exosomes are isolated to some degree and the cancer-specific exosome-associated marker is then analyzed. Thus one embodiment of the invention provides a method of diagnosing a cancer in a patient comprising (1) isolating exosomes from a sample obtained from a patient and (2) determining the status of a cancer-type marker associated with the exosomes isolated in (1); wherein an abnormal status of the marker in (2) indicates the presence of indicates the presence of a specific cancer type, or cancer in a specific tissue or organ, in the patient. Examples of cancer-type markers include some of those listed in Table 1 (e.g., EGFRvIII, ErbB2). An example of such an analysis is given in FIG. 2.

As used herein, “cancer type” means a cancer in or originating from a particular tissue or organ and/or a cancer with a particular molecular or clinical feature. Often, the specificity of the “cancer type” varies with the application, including tissue type (e.g., squamous versus cuboidal), organ type (e.g., breast versus lung), and clinical subtype (e.g., triple-negative breast cancer). For example, finding certain markers (e.g., surfactant proteins B [SFTPB] and C [SFTPC]) on exosomes derived from a tumor can indicate that it is a lung tumor. See, e.g., Johnson et al., J. BIOL. CHEM. (2001) 276:14658-14664. Breast cancer cells can show overexpression of HER2, which in turn correlates with a particular clinically-defined subtype of breast cancer. Knowing that the patient has breast cancer will generally prompt a physician to treat in a particular way (e.g., surgery, hormone therapy, and optional adjuvant chemotherapy), while the additional knowledge that the patient has HER2-overexpressing breast cancer can prompt further personalization of that treatment (e.g., adding trastuzumab).

The present invention provides various methods of determining what cancer type a patient has. For example, cancer type may be determined by more traditional diagnostic methods such as imaging (e.g., CAT scan, MRI, X-ray, etc.), physical examination (e.g., digital rectal examination—DRE—in prostate cancer), biopsy, etc. Thus, one aspect of the invention provides a method of determining whether a patient has a specific cancer type, comprising determining the status of exosomes and/or an exosome-associated marker in a sample obtained from the patient, wherein an abnormal status of such exosomes and/or the exosome-associated marker indicates the presence of cancer, and performing a physical examination, imaging test, or biopsy to determine the specific cancer type.

Alternatively, molecular diagnostics may give cancer-type specificity. For example, the cancer marker in the above embodiments may simultaneously be a cancer-type marker. As used herein, “cancer-type marker” means a biomarker whose status is correlated with the presence of a specific cancer type. One example is prostate-specific antigen (PSA), where a status of elevated levels is correlated with prostate cancer. Thus one embodiment provides the quantification of PSA-bearing exosomes as a substitute for measuring PSA in the serum, which may be a more clinically accurate measure of cancer-relevant PSA. Another antigen of particular interest is CA-125. It is frequently expressed in ovarian tumors. It is also expressed at lower levels in normal individual's serum, but since such individuals either don't have exosomes or have far fewer of them, comparing CA-125-bearing exosomes in individuals with tumors compared to controls could be much more effective than comparing levels of CA-125 free in serum. CA-125 is somewhat non-specific for tumor type in that it is expressed in gynecological cancers other than ovarian and in colon cancer. Thus elevated levels of CA-125-bearing exosomes could indicate the value of more expensive and invasive tests such as an MRI of the abdominal/pelvic region, a pelvic exam, a colonoscopy and possibly biopsies. Yet another example is EGFRvIII, whose expression is correlated with a particular form of glioblastoma (i.e., particular mutated EGFR status is correlated with a specific clinical subtype of glioblastoma). Those skilled in the art are familiar with additional examples (e.g., HER2, EGFR, KRAS, etc.).

Another option to specify the cancer type is by determining that the patient has cancer and then, through the same or a subsequent assay, determining the tissue of origin using a tissue-specific marker. As used herein, a “tissue-specific marker” is a biomarker whose status is correlated with a specific tissue or organ type, though not necessarily with cancer. Several examples of such tissue-specific markers, as well as the tissues and/or organs with which they are correlated, are given in FIG. 2. This additional marker is generally, though not necessarily, exosome-associated.

Thus, one aspect of the invention provides a method of determining whether a patient has a specific cancer type, comprising determining the status of exosomes and/or an exosome-associated marker (e.g., those listed in Table 1) in a sample obtained from the patient and determining the status of at least one exosome-associated tissue-specific marker in a sample obtained from the patient, wherein an elevated level of exosomes and/or the exosome-associated marker indicates the patient has cancer and wherein a particular status of the exosome-associated tissue-specific marker indicates the patient has a specific cancer type. In the context of tissue-type markers, status will often, though not necessarily, mean level (i.e., presence, absence and/or amount). Often a particular biomolecule is exclusively or predominantly found in or produced by a particular tissue type or organ. Examples include EpCAM (epithelial tissue), SFTPB (lung epithelium), PSA (prostate tissue), etc. As another example, GPA33 (glycoprotein A33 [transmembrane]) is expressed in the epithelium of the colon and small intestine. See, e.g., Heath et al., PROC. NATL. ACAD. SCI. USA (1997) 94:469-474. Thus, one skilled in the art could, according to the present invention, determine the level of exosomes (e.g., by determining the level of one or more of the exosome-associated markers listed in Table 1) and also determine whether or to what extent (e.g., what proportion of) such exosomes also bear the GPA33 antigen. If exosome levels are elevated and some significant proportion of these exosomes also bear the GPA33 antigen, then the patient has colon cancer. In other embodiments for example, determining that exosome levels are high and PSA is present (particular PSA “status”) will indicate the patient has prostate cancer. Other times, particular biomolecules are upregulated in specific tissue types or organs (i.e., found in higher levels in some tissues or organs as compared to others).

There is a large and ever growing catalog of exosome-associated, cancer-type, and tissue-specific markers. Further, those skilled in the art will appreciate that these categories are not mutually exclusive (e.g., an exosome-associated marker may also be tissue-specific). Thus one aspect of the invention provides methods comprising determining the status of a panel of markers. In some embodiments, the invention provides a method of determining whether a patient has cancer comprising determining the status of a panel of exosome-associated markers in a sample obtained from the patient, wherein a particular status of the panel of markers indicates the patient has cancer. In some embodiments, the invention provides a method of determining whether a patient has a specific cancer type, comprising determining the status of a panel of cancer-type markers in a sample obtained from the patient, wherein a particular status of the panel of markers indicates the patient has a specific cancer type. In yet other embodiments, the invention provides a method of determining whether a patient has a specific cancer type, comprising determining the status of exosomes and/or an exosome-associated marker in a sample obtained from the patient and determining the status of a panel of tissue-specific markers in a sample obtained from the patient, wherein an abnormal status of exosomes and/or the exosome-associated marker indicates the patient has cancer and wherein a particular status of the panel of markers indicates the patient has a specific cancer type (see, e.g., FIG. 2).

In some embodiments the panel of markers comprises 2, 3, 4, 5, 6, 7, 8, 9, 10, 11, 12, 13, 14, 15, 16, 17, 18, 19, 20, 25, 30, 35, 40, 45, 50, 60, 70, 80, 90, 100, or more markers. The markers may be any exosome-associated markers, including cancer-type markers, or tissue-type markers or a combination of these. In some embodiments the panel of markers comprises two or more markers listed in Table 1. In some embodiments the panel of markers comprises two or more markers shown in FIG. 2, wherein the presence or absence (or abnormal status) of specific markers indicates, according to the flowcharts in FIG. 2, the patient has cancer of a specific type. In some embodiments the panel comprises at least two markers chosen from those listed in Table 1.

In further embodiments the status of individual markers in the panel is tested in a certain order in order to narrow down which specific cancer type is present. One example is illustrated in FIG. 2A-2D. Specifically, when elevated exosome levels are found in a patient's sample, one may also test the sample for the status of cytokeratin 7 (CK7) and cytokeratin 20 (CK20) followed by various other markers. If both CK7 and CK20 are absent as in FIG. 2A [110], then PSA, PSAP, PSMA, Hep Par 1, AFP, CAM 5.2, CD10, Vimentin, RCC, and EMA (or any combination thereof or any single marker) may be tested [210] to determine the specific organ/tissue of origin. If PSA, PSAP, and/or PSMA are found, then the cancer is prostate adenocarcinoma [310]. If Hep Par 1, AFP, and/or CAM 5.2 are present, then the cancer is hepatocellular carcinoma [311]. If CD10, Vimentin, RCC, and/or EMA are present, then the cancer is renal cell carcinoma (clear cell type) [312].

If CK7 is absent and CK20 is present as in FIG. 2B [120], then Ae1/3, CAM 5.2, CK19, CEA (polyclonal), and EMA (or any combination thereof or any single marker) may be tested [220] to confirm that the cancer is colon adenocarcinoma. If any of these markers is found, then the cancer is colon adenocarcinoma [320]. Imaging and/or endoscopy may be performed [420] either in place of the additional marker tests [320] or as an additional confirmation.

If CK7 is present and CK20 is absent as in FIG. 2C [130], then PSA, PSAP, PSMA, Thyroglobulin, Calictonin, HER2, GCDFP-15, Chromogranin, Synaptophysin, CD56, (NCAM), Leu7, CK5/6, CEA, Mucicarmine, B72.3, Leu, M1, (CD15), Calretinin, HBME-1, Mesothelin and Vimentin (or any combination thereof or any single marker) may be tested [230] to determine the specific organ/tissue of origin. If PSA, PSAP, and/or PSMA are found, then the cancer is prostate cancer [330]. If Thyroglobulin and/or Calictonin are present, then the cancer is thyroid cancer [331]. If HER2 and/or GCDFP-15 are found, then the cancer is breast cancer [332]. If Chromogranin, Synaptophysin, CD56, (NCAM), and/or Leu7 are found, then the cancer is small cell/neuroendocrine carcinoma of the lung [336]. If CK5/6 is found, then the cancer may be squamous cell carcinoma of the lung [337] (diagnosis may be confirmed by imaging [430]). CEA, Mucicarmine, B72.3, and/or Leu M1 (CD 15) are found, then the cancer may be adenocarcinoma of the lung [338] (diagnosis may be confirmed by imaging [430]). If Calretinin, HBME-1, CK5/6, and/or Mesothelin are found, then the cancer may be mesothelioma [333] (if the only marker found is CK5/6, imaging [430] may be necessary). If Vimentin is found, then the cancer is endometrial cancer [334]. If CK5/6 and/or CEA are found, then the cancer may be cervical cancer [332] (confirmation, e.g., by pap smear, may be necessary since these markers are also expressed by other CK7+/CK20− tissue types).

If CK7 and CK20 are both present as in FIG. 2D [140], then CA-125, Mesothelin, 34βE12, Villin, Uroplakin III, and/or CD10 (or any combination thereof or any single marker) may be tested [240] to determine the specific organ/tissue of origin. If CA-125 and/or Mesothelin are found, then the cancer may be ovarian carcinoma [340] (confirmation, e.g., by imaging, may be necessary since CA-125 is also expressed in other CK7+/CK20+ tissues). If 34βE12, Villin, and/or CA-125 are present, then the cancer may be cholangio carcinoma (bile duct cancer) [341] (confirmation, e.g., by imaging, may be necessary since CA-125 is also expressed in other CK7+/CK20+ tissues). If Uroplakin III is found, then the cancer is urothelial carcinoma [342]. If CD10 is found, then the cancer is papillary-type renal cell carcinoma [343]. If no marker is found, then the cancer may be chromophobe renal cell carcinoma [344] (diagnosis may be confirmed microscopically).

In some patients, a diagnosis of the desired specificity requires more than simply determining the status of exosomes and/or an exosome-associated marker. Many physiological conditions other than cancer are characterized by increased exosome secretion, including rheumatoid arthritis. For example, as mentioned above, epithelial exosomes are elevated in pregnant women and in patients with diabetic nephropathy. In many cases, therefore, alternative conditions must be ruled out before a diagnosis of cancer can be made. This may require additional tests specific for the given alternative condition, e.g., a pregnancy test.

Thus, one aspect of the invention provides a method for determining whether a patient has cancer comprising determining the status of exosomes and/or an exosomes-associated marker in a sample from this patient and determining whether the patient has a non-cancerous condition characterized by increased exosome secretion, wherein an abnormal status of exosomes (e.g., EpCAM-bearing exosomes, CD63-bearing exosomes, etc.) and/or the exosome-associated marker and the absence of any non-cancerous condition characterized by increased exosome secretion indicates the patient has cancer. Examples of how one might determine whether a patient has a noncancerous condition characterized by increased exosome secretion include, but are not limited to, determining whether the patient is pregnant (e.g., by a pregnancy test), determining whether the patient is diabetic (or determining whether the patient shows signs of diabetic nephropathy), etc.

Alternatively, one may rule out other conditions by differentiating tumor-derived exosomes, referred to herein variously as oncosomes or cancer-derived exosomes, from exosomes derived from some other cell. Such oncosomes are especially useful according to the present invention in detecting, classifying and monitoring cancer since they are particularly prevalent in epithelial cancers such as those of the lung, colon, breast, prostate, ovaries, endometrium, etc. One way to differentiate oncosomes from noncancerous exosomes is by determining the status of some exosome-associated biomarker. Generally, the methods of the invention will involve determining whether a particular marker has an abnormal status, when such abnormal status is somehow associated with cancer cells. Determining whether a marker's status is abnormal in a sample will generally comprise comparing the marker's status in the patient's sample with the marker's index or reference status (e.g., index values discussed above) in an average, normal, or healthy patient's sample.

Another aspect of the invention provides a method of screening for cancer in a patient comprising identifying a patient at risk of having, or in need of screening for, cancer and determining the status of exosomes and/or an exosomes-associated marker in a sample obtained from the patient, wherein an abnormal status of exosomes and/or an exosomes-associated marker in the sample indicates the presence of cancer. Patients may be identified as at risk of having, or in need of screening for, cancer in a variety of ways and based on numerous clinical and/or molecular characteristics. One class of patients at risk of having cancer and in need of screening is those patients known to carry a germline deleterious mutation in a tumor suppressor gene. Examples include, but are not limited to, BRCA1, BRCA2, PTEN, p16, MLH1, MSH6, APC, MYH, etc. In such patients, cancer-type specificity is often less crucial since, for example, a BRCA1-mutant patient whose exosome levels indicate cancer would be expected have breast or ovarian cancer rather than some other type of cancer. However, if desired, additional tests to determine the type of cancer may be performed as discussed above. Age and environmental factors may also define at-risk patients in need of screening. For example, prostate cancer is overwhelmingly found in men over 50 years of age. Thus, men over 50 may be considered patients at risk of having, or in need of screening for, prostate cancer. The relatively non-invasive nature of blood, plasma, or serum detection (i.e., simple blood draw) makes such widespread screening attractive and practical.

Thus in some embodiments the invention provides a method of detecting cancer comprising identifying a patient having a mutation in a gene selected from the group consisting of BRCA1, BRCA2, PTEN, p16, MLH1, MSH6, APC, and MYH; and determining the status of exosomes and/or an exosomes-associated marker in a sample obtained from the patient; wherein an abnormal status of exosomes and/or an exosomes-associated marker in the sample indicates the presence of cancer. In some such embodiments the method further comprises additional tests to determine/confirm which type of cancer is present.

Yet another aspect of the invention provides a method of detecting recurrence in a cancer patient comprising providing a sample obtained from the patient and determining the status of exosomes and/or an exosomes-associated marker in the sample, wherein an abnormal status of exosomes and/or an exosomes-associated marker in the sample indicates recurrence. Because it is difficult to remove or kill all cancerous cells, one of the main challenges in cancer treatment is making sure a cancer removed by surgery and/or treated with drugs has not returned. Thus this aspect of the invention is particularly useful in monitoring cancer patients following treatment. Much like the at-risk patients discussed above, cancer-type specificity is not crucial. If a lung cancer patient is found to have increased exosome levels in his blood, plasma or serum several months or years after treatment, then the new cancer is likely to be a return of the former lung cancer. As above, in some embodiments further testing (e.g., imaging) to confirm the type of cancer or to characterize the cancer (e.g., stage) is encompassed by the invention. In some embodiments exosome levels are measured soon after treatment (e.g., to determine a post-treatment baseline) and then monitored at regular intervals there after in order to catch any significant increase (e.g., from this baseline).

Still another aspect of the invention provides a method comprising identifying a patient who is a candidate for biopsy and determining the status of exosomes and/or an exosomes-associated marker in a sample obtained from the patient, wherein an abnormal status of exosomes and/or an exosomes-associated marker in the sample indicates a biopsy is desirable. Biopsies are often expensive, painful, and time-consuming and, furthermore, most biopsies are negative for cancer. Thus this aspect of the invention provides an effective tool which could allow one to know whether a biopsy is required (e.g., after PSA, mammography, etc.). As used herein, “candidate for biopsy” refers to a patient suspected of having cancer, for whom a biopsy could be expected to confirm the presence of such cancer. For example, if a patient's mammography indicates the presence of an abnormal mass, methods according to this aspect of the invention can help determine whether the mass is likely to be cancerous and thus determine whether the discomfort and expense of a biopsy is warranted.

As mentioned above, some embodiments of the invention involve exosome analysis combined with more traditional diagnostic techniques. For example, physical examination (e.g., digital rectal exam for prostate cancer), imaging (e.g., mammography), and/or biopsy may be used to confirm a diagnosis indicated by exosome analysis according to the invention. Alternatively, such techniques may be combined with exosome analysis to yield a more comprehensive diagnosis. As an illustrative example, an exosome screen may indicate the presence of cancer in a patient and these exosomes may be found to be CK7+/CK20− and have the marker CK5/6 associated with them. One may not be able to conclusively determine based solely on this information whether the cancer is squamous cell carcinoma of the lung, cervical cancer, or mesothelioma at some unknown organ (see FIG. 2C). Thus, a physician may take the further step of imaging to pinpoint the location of the cancer (e.g., in or near the lung). The physician may further perform a biopsy to determine whether the cancer is squamous cell carcinoma of the lung or cancer of the mesothelial lining of the lung.

Various techniques can be used to enrich for exosomes and/or exosome-associated markers and to determine the status of exosomes and/or exosome-associated markers in a sample. Examples of enrichment techniques include, but are not limited to: flow cytometry (i.e., cell sorting); centrifugation (e.g., ultracentrifugation); physical filtration; affinity chromatography; contacting a sample with a solid surface (e.g., plate, well, bead, etc.) containing an antibody against an exosome-associated marker in order to capture any exosomes on the surface; etc. or any combination of these. Example 5 describes one technique using ultracentrifugation combined with filtration and ECL to enrich a sample. As used herein, “ultracentrifugation” has its conventional meaning in the art. In some embodiments ultracentrifugation comprises spinning the sample at least 20,000×g, at least 30,000×g, at least 40,000×g, at least 50,000×g, at least 75,000×g, at least 100,000×g, at least 150,000×g, at least 250,000×g, at least 500,000×g, at least 750,000×g, or at least 1,000,000×g. Those skilled in the art will appreciate that several of the variables in the Examples can be adjusted while still allowing for enrichment. Various suitable sizes and types of filtration may be used, including filtering to elute exosome-sized particles in the sample (e.g., >100 nm, 150 nm, 200 nm, 250 nm), filtering to capture exosome-sized particles (e.g., <50 nm, 40 nm, 30 nm, 20 nm, 10 nm), or both combined.

Various techniques can be used to determine the status of exosomes and/or an exosome-associated marker in a patient sample including, but are not limited to: flow cytometry; enzyme-linked immunosorbent assay (ELISA) and its numerous variations (e.g., electrochemiluminescence or “ECL”); immunohistochemistry (IHC); etc. or any combination of these. As can be seen, determining the status of exosomes and/or exosome-associate markers will often also involve some level of enrichment.

For example, flow cytometry can both sort exosomes (i.e., separate exosomes of interest out of the sample milieu) and quantitate them, as shown in Example 1. Flow cytometry is well-suited to the methods of the invention since multiple markers may be assayed at once by analyzing a panel of antigens by cell sorting using antibodies to each antigen and counting on a multichannel sorter. Thus the invention provides a method of quantitating exosomes comprising isolating exosomes from a patient sample and counting the exosomes using flow cytometry. In some embodiments the isolation and counting may be done simultaneously, such as by using fluorescent-activated cell sorting (FACS) adapted for use with exosomes.

Similarly, ECL ELISA can enrich a sample for exosomes and/or exosome-associated markers by capturing these on a solid surface (using, e.g., integrins, antibodies, etc.) and can then determine their level by way of a separate detection reagent (e.g., integrins, antibodies, etc.). See Example 4. Thus the invention provides a method of detecting cancer comprising fixing exosomes and/or an exosome-associated marker to a solid surface and determining the level of at least one exosome-associated marker, wherein an abnormal status of the exosome-associated marker indicates an increased likelihood of cancer. In some embodiments the exosomes and/or exosome-associated markers are fixed to the solid surface by way of a linker. In some embodiments the linker specifically binds exosomes and/or an exosome-associated marker (e.g., integrins, antibodies such as anti-CD63, anti-CD36, or any antibody against any marker listed in Table 1, etc.). Alternatively, the solid surface can be “sticky”—i.e., the surface may be made of a material that itself binds exosomes and/or exosome-associated markers (e.g., latex, graphite).

In some embodiments IHC is used to determine the status of exosomes and/or an exosome-associated marker. This will often be in connection with exosome blocks. Thus the invention provides a method of detecting cancer comprising determining the status of an exosome-associated marker by contacting an exosome block sample from a patient with at least one antibody that specifically binds the marker, wherein an abnormal status for the marker indicates an increased likelihood of cancer.

Though much of the preceding discussion has focused on exosome-surface markers, the invention is not so limited as exosome-associated markers may also be found within an exosome's interior. Examples include proteins, mRNA, microRNA, DNA, etc. Various techniques exist to analyze the interior exosome-associated markers. To analyze protein markers, immunological techniques are available including, but no limited to, intraexosomal staining flow cytometry (see Example 1 below), ELISA (see Example 2 below), IHC (e.g., using exosome blocks such as in Example 3 below), etc. To analyze a nucleic acid marker, the exosomes may be solubilized to release their contents and nucleic acids of interest may be quantitated (e.g., qPCR, microarray), genotyped (e.g., TaqMan®), sequenced, etc. Alternatively, fluorescence in situ hybridization (FISH) may be adapted to analyzing exosome-associated nucleic acid markers (especially using exosome blocks).

Another aspect of the invention provides exosome blocks. These blocks may be prepared in a manner similar to formalin-fixed paraffin-embedded (FFPE) tissue samples, the main difference being that isolated exosomes rather than excised tissue form the basis of exosome blocks. Pathologists routinely make cell pellets from formalin-fixed body fluid samples and embed them in paraffin to produce a cell block. Likewise, one may take the fixed, pelleted exosomes and embed them in paraffin to make an exosome block. This tissue block can then be sliced (e.g., with a microtome) and sections put on slides, or a tissue microarray can be constructed. The interior of many of the exosomes will be exposed when the exosome block is sliced, and any analysis aimed at interior exosome-associated markers (e.g., IHC with cytoplasmic antibodies) can be performed to, e.g., determine the tissue of origin of the exosome pellet. Thus the invention provides an exosome block comprising formalin-fixed exosomes embedded in paraffin.

The results of these and any other analyses according to the invention will often be recorded and communicated to physicians, genetic counselors and/or patients (or other interested parties such as researchers) in a transmittable form that can be communicated or transmitted to any of the above parties. Thus one aspect of the invention provides a method comprising determining the status of exosomes and/or an exosome-associated marker in a sample obtained from a patient, recording and/or communicating whether the status is abnormal, and recording and/or communicating that an abnormal status indicates the presence of cancer. In one embodiment the method comprises determining the level of exosomes in a sample obtained from a patient and, if the level of exosomes is increased, recording and/or communicating that the patient has an increased likelihood of cancer.

Transmittable forms for communicating the results of a test can vary and can be tangible or intangible. The results can be embodied in descriptive statements, diagrams, photographs, charts, images or any other visual forms. For example, graphs showing exosome level information can be used in explaining the results. Diagrams showing such information for additional markers are also useful in indicating some testing results. The statements and visual forms can be recorded on a tangible medium such as papers, computer readable media such as floppy disks, compact disks, etc., or on an intangible medium, e.g., an electronic medium in the form of email or website on Internet or intranet. In addition, results can also be recorded in a sound form and transmitted through any suitable medium, e.g., analog or digital cable lines, fiber optic cables, etc., via telephone, facsimile, wireless mobile phone, internet phone and the like.

Thus, the information and data on a test result can be produced anywhere in the world and transmitted to a different location. As an illustrative example, when an exosome level assay is conducted outside the United States, the information and data on a test result may be recorded or generated, cast in a transmittable form as described above, and then imported into the United States. Accordingly, the present invention also encompasses a method for producing a transmittable form of information on exosome (and/or exosome-associated marker) status for at least one patient sample. The method comprises the steps of (1) determining exosome (and/or exosome-associated marker) status according to the methods of the present invention; and (2) embodying the result of the determining step in a transmittable form. The transmittable form is the product of such a method.

Techniques for analyzing such expression, activity, and/or sequence data (indeed any data obtained according to the invention) will often be implemented using hardware, software or a combination thereof in one or more computer systems or other processing systems capable of effectuating such analysis.

One aspect of the invention provides methods of treating a patient comprising determining the status of exosomes and/or an exosomes-associated marker as discussed above. The treatment methods of the invention generally further comprise some action based on the exosome (and/or exosome-associated marker) status determination. Examples of actions based on the exosome determination include recommending a biopsy; prescribing a particular treatment; administering a particular therapeutic composition; prescribing, recommending, or performing surgery; monitoring for more signs of malignancy (e.g., recommending an additional test for cancer), etc. Other actions (such as communicating, e.g., a particular diagnosis or prognosis to a patient) are also contemplated in the treatment methods of the invention as discussed above.

In another aspect of the present invention, a kit is provided for practicing the methods of the present invention. The kit may include a carrier for the various components of the kit. The carrier can be a container or support, in the form of, e.g., bag, box, tube, rack, and is optionally compartmentalized. The carrier may define an enclosed confinement for safety purposes during shipment and storage.

The kit also includes at least one component useful in determining the status of exosomes and/or an exosomes-associated marker using any of the detection techniques discussed herein. For example, the kit many include probes to exosome-associated markers (e.g., anti-CD63 antibodies) or probes to a panel of markers useful for differentiating tissue or cancer types.

Various other components useful in the detection techniques may also be included in the detection kit of this invention. Examples of such components include, but are not limited to, Taq polymerase, deoxyribonucleotides, dideoxyribonucleotides other primers suitable for the amplification of a target DNA sequence, RNase A, and the like. In addition, the detection kit preferably includes instructions on using the kit for practicing the methods of the present invention using human samples.

### EXAMPLES

### Example 1

### Detection/Quantitation/Characterization of Exosomes by Flow Cytometry

Exosomes may also be measured by flow cytometry. Flow cytometry is generally used to measure cell-surface antigen expression and thus may be readily adapted to measuring antigens on the surface of exosomes. In general, exosomes are isolated from a sample (e.g., serum) and then incubated with a labeled antibody (e.g., fluorescence-labeled) against some surface marker expected to be found on the exosomes (e.g., EpCAM). This solution is then passed through a cell sorter, which detects and “counts” the number of labeled exosomes as they flow through an optical/electronic detection apparatus. Multiple labeled antibodies may be used in the same sample as cell sorters are capable of detecting multiple wavelengths of light at once. Flow cytometry may also be used to detect and quantitate cytoplasmic antigens. For this it is generally necessary to fix and permeabilize cells to enable antibodies to gain access to them, after which the process is generally the same as above.

The experiment detailed below has demonstrated that capturing exosomes using anti-CD63 or anti-EpCAM antibodies and quantitating them using flow cytometry can differentiate ovarian cancer (OVCA) patients from non-cancerous controls.

Plasma samples were obtained from eight patients with no cancer and from eight OVCA patients. These plasma samples were subjected to differential centrifugation to enrich for exosomes as follows and then protein was quantified by the Bradford Assay (Bradford, A Rapid and Sensitive Method for the Quantitation of Microgram Quantities of Protein Utilizing the Principle of Protein-Dye Binding, ANAL. BIOCHEM. (1976) 72:248-254):
1. Spun at 2000×g for 30 minutes—transferred supernatant to new tube (contained exosomes)
2. Spun at 12000×g for 45 minutes—transferred supernatant to new tube (contained exosomes)
3. Spun at 110,000×g for 2 hours (ultracentrifugation)—pellet contained the exosomes
4. Resuspended pellet in 1 ml PBS
5. Filtered through 0.2 μm filter
6. Spun at 110,000×g for 1 hour
7. Resuspended in 100 μl PBS

TABLE 2

| | mg/ml by Bradford | μl for 14 μg | PBS |
|---|---|---|---|
| OVCA Sample # | | | |
| 29449 | 1.09 | 12.8 | 87.2 |
| 31182 | 0.79 | 17.7 | 82.3 |
| 31216 | 0.77 | 18.2 | 81.8 |
| 32455 | 0.61 | 23.0 | 77.0 |
| 38704 | 0.62 | 22.6 | 77.4 |
| 39004 | 0.14 | 100.0 | 0.0 |
| 41365 | 0.44 | 31.8 | 68.2 |
| 41757 | 0.83 | 16.9 | 83.1 |
| Normal Sample # | | | |
| 39270 | 1.49 | 9.4 | 90.6 |
| 39271 | 1.21 | 11.6 | 88.4 |
| 42201 | 0.79 | 17.7 | 82.3 |
| 42204 | 1.01 | 13.9 | 86.1 |
| 42205 | 0.75 | 18.7 | 81.3 |
| 42208 | 0.45 | 31.1 | 68.9 |
| 42209 | 0.54 | 25.9 | 74.1 |
| 42214 | 0.36 | 38.9 | 61.1 |

Latex beads were coated with anti-CD63 antibodies and then contacted/incubated with the exosome samples isolated above as follows:
1. Incubated 2.0 μg of BD Pharmingen anti-CD63 antibody with 5 μl Aldehyde latex bead 15 minutes at room temperature.
2. Brought volume up to 500 μl with PBS.
3. Incubated overnight at 4° C. with Rotor.
4. Added 550 μl 1M glycine in PBS and incubate 30 minutes at room temperature.
5. Washed 3 times with PBS/0.5% BSA.
6. Incubated with 14 μg plasma pellet for 2 hours at room temperature.
7. Washed 3 times with PBS/0.5% BSA.
8. Resuspended in 250 μl PBS/0.5% BSA.

The resulting solution was then incubated with phycoerythrin (PE)-conjugated anti-CD63 antibody (CD63 BD Pharmingen Clone #H5C6) and finally run through an Accuri C6 cell-sorter set to detect PE (488 nm laser [excitation] and 585/40 nm filter for PE). Isotype control samples comprising beads with PE-conjugated IgG1 antibodies were also run through the cell sorter under identical conditions. The fluorescence values (Median FL2-A) for these samples are shown in Table 3 below:

TABLE 3

| | Plasma | | | | |
|---|---|---|---|---|---|
| | Exosome | | PE-Isotype | | |
| | Coated | PE-cd63 | (IgG1) | Cell | |
| | Latex Bead | 0.013 mg/ml | 0.2 mg/ml | Staining | Median |
| Well | (10 μl) | Bdpharmigen | BDpharmingen | Buffer | FL2-A |
| 1 | 29449 | 20 μl | | 50 μl | 128 |
| 2 | 31182 | 20 μl | | 50 μl | 190 |
| 3 | 31216 | 20 μl | | 50 μl | 128 |
| 4 | 32455 | 20 μl | | 50 μl | 372 |
| 5 | 38704 | 20 μl | | 50 μl | 263 |
| 6 | 39004 | 20 μl | | 50 μl | 147 |
| 7 | 41365 | 20 μl | | 50 μl | 526 |
| 8 | 41757 | 20 μl | | 50 μl | 132 |
| 9 | 39270 | 20 μl | | 50 μl | 125 |
| 10 | 39271 | 20 μl | | 50 μl | 127 |
| 11 | 42201 | 20 μl | | 50 μl | 137 |
| 12 | 42204 | 20 μl | | 50 μl | 124 |
| 13 | 42205 | 20 μl | | 50 μl | 128 |
| 14 | 42208 | 20 μl | | 50 μl | 128 |
| 15 | 42209 | 20 μl | | 50 μl | 127 |
| 16 | 42214 | 20 μl | | 50 μl | 137 |
| 1 | 29449 | | 1.3 μl | 68.7 μl | 120 |
| 2 | 31182 | | 1.3 μl | 68.7 μl | 121 |
| 3 | 31216 | | 1.3 μl | 68.7 μl | 122 |
| 4 | 32455 | | 1.3 μl | 68.7 μl | 119 |
| 5 | 38704 | | 1.3 μl | 68.7 μl | 118 |
| 6 | 39004 | | 1.3 μl | 68.7 μl | 121 |
| 7 | 41365 | | 1.3 μl | 68.7 μl | 120 |
| 8 | 41757 | | 1.3 μl | 68.7 μl | 120 |
| 9 | 39270 | | 1.3 μl | 68.7 μl | 123 |
| 10 | 39271 | | 1.3 μl | 68.7 μl | 123 |
| 11 | 42201 | | 1.3 μl | 68.7 μl | 119 |
| 12 | 42204 | | 1.3 μl | 68.7 μl | 120 |
| 13 | 42205 | | 1.3 μl | 68.7 μl | 118 |
| 14 | 42208 | | 1.3 μl | 68.7 μl | 123 |
| 15 | 42209 | | 1.3 μl | 68.7 μl | 124 |
| 16 | 42214 | | 1.3 μl | 68.7 μl | 120 |

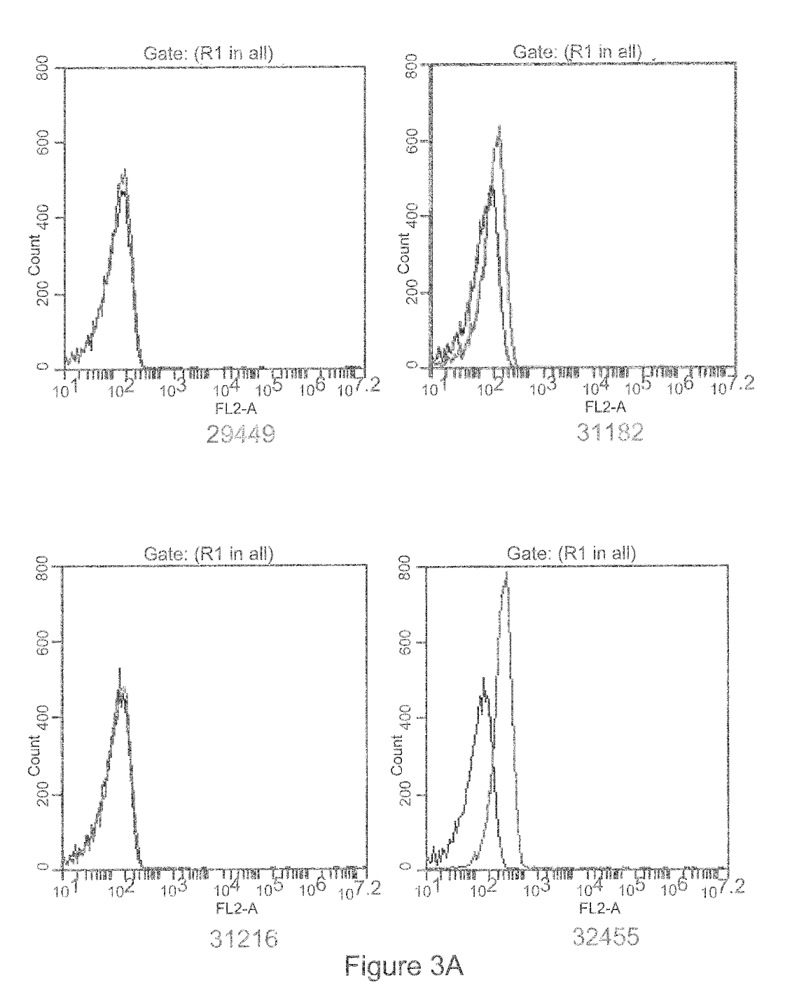

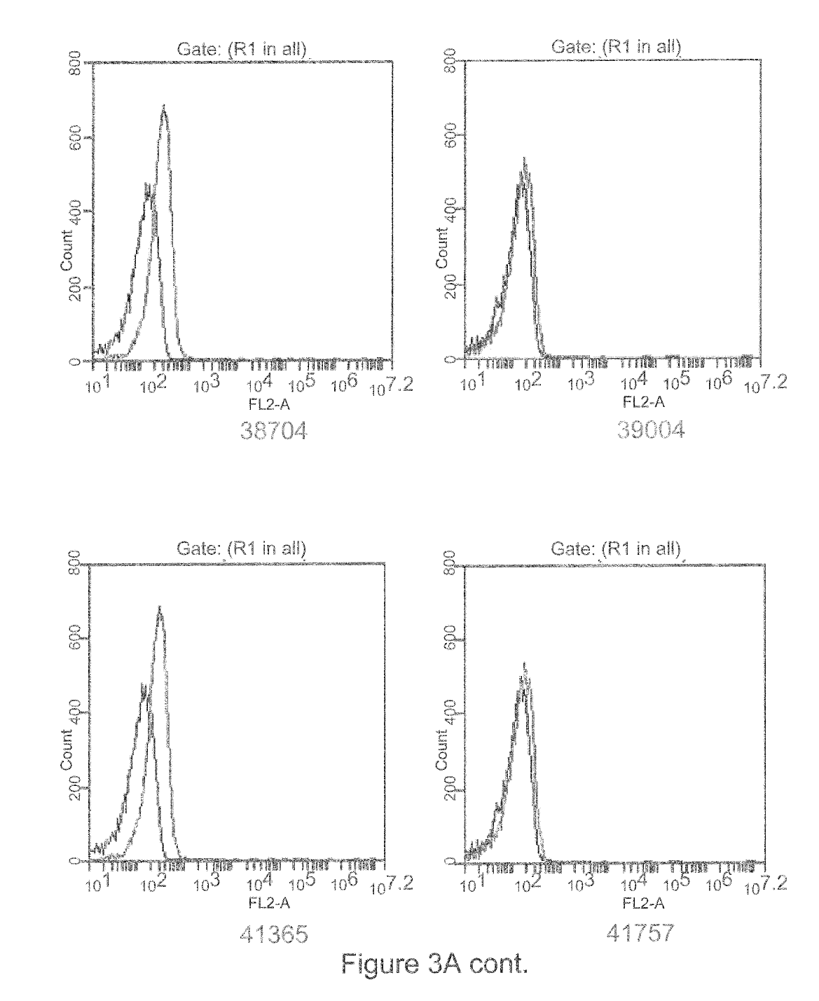

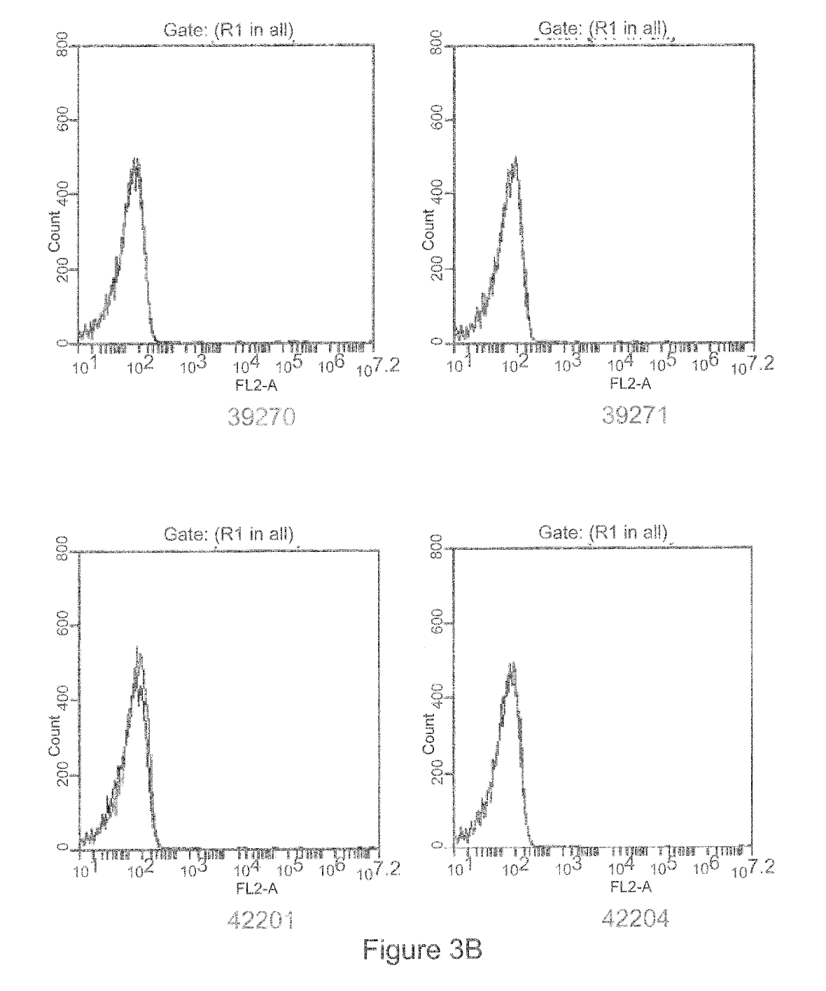

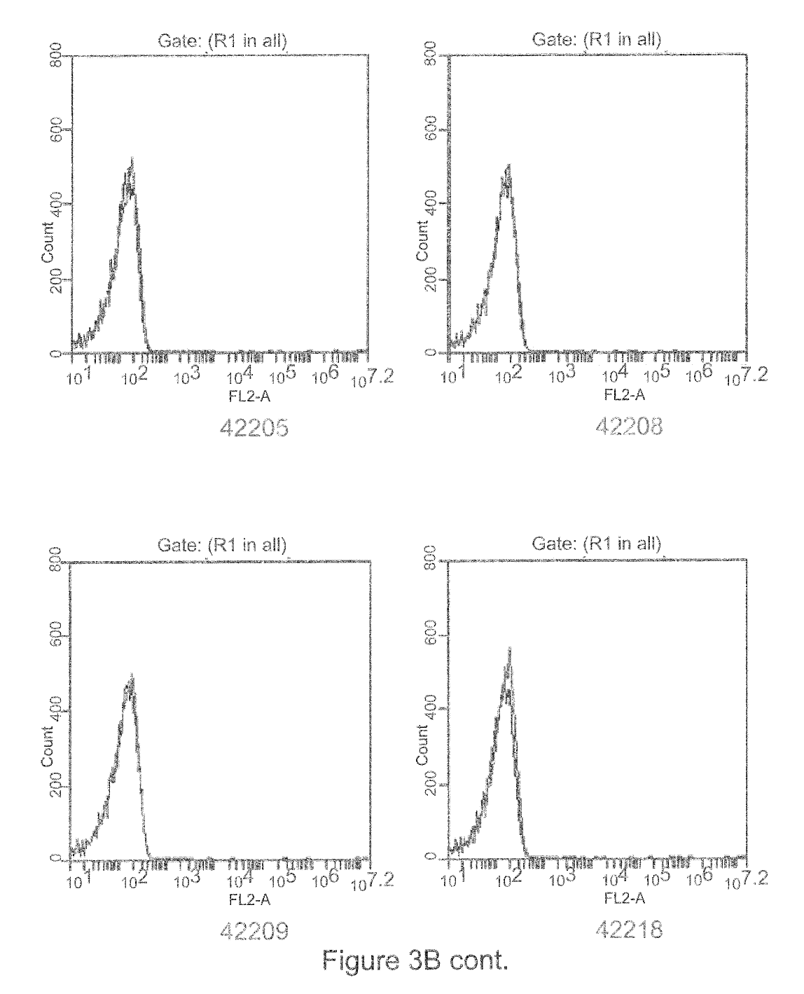

As seen in FIG. 3A, fluorescence was shifted substantially higher in 4 out of 8 OVCA patients (31182, 32455, 38704, and 41365), with some shift in an additional two OVCA samples (39004, 41757). In contrast, in FIG. 3B no significant shift was seen in any of the non-cancer controls (39270, 39271, 42201, 42204, 42205, 42208, 42209, and 42214).

### Example 2

### Detection/Quantitation/Characterization of Exosomes by ELISA

ELISA (Enzyme-Linked immunosorbent assay) can be adapted to detect, quantify, and characterize exosomes according to the invention. The purpose of an ELISA is to determine if a particular protein is present in a sample and optionally, how much. EpCAM-bearing exosomes were detected and quantitated in the supernatant of epithelial cancer cell lines in the following experiment.

Reagents:
1. Capture antibody is a mouse monoclonal (clone 158210, R&D systems catalog #MAB 960) raised against the extracellular domain of Human EpCAM.
2. Detection antibody is a goat polyclonal (R&D systems catalog #BAF960) raised against recombinant extracellular domain of Human EpCAM.
3. Quantification achieved by comparison with a standard curve generated using recombinant extracellular domain of Human EpCAM (recombinant EpCAM/Fc chimera, R&D catalog #960-EP-050).

Sample Preparation:
1. Exosomes were isolated by differential centrifugation and filtration using the same process described in Example 1.
2. Pellets from final ultracentrifuge spin were resuspended in PBS and measured for total protein using the Bradford assay prior to addition to ELISA assay.
3. Cell culture supernatants and supernatants from ultracentrifugation were added directly to 96-well plates pre-coated with the capture antibody for the assay:
   a. Detection antibody was conjugated to biotin.
   b. Detection enzyme was Horseradish-peroxidase conjugated to streptavidin (R&D cat#DY998).
   c. Enzyme substrate was H2O2+tetramethylbenzidine (R&D cat#DY999).
   d. The ELISA was read on a Packard Bioscience Fusion microplate reader set at 450 nM.

Experiment:

Four epithelial cancer cell lines (SKOV-3, OVCa-5, MCF-7, and HCT-15) have been evaluated by ELISA for presence of EpCAM-bearing exosomes. EpCAM was measured by ELISA (1) in the Cell-conditioned media and (2) in the Cell-conditioned media depleted by centrifugation, and (3) total protein was also measured in the centrifugation pellet. Results are summarized in Table 4 below:

TABLE 4

| | SKOV-3 | OVCa-5 | MCF-7 | HCT-15 |
|---|---|---|---|---|
| Medium | 314 pg/ml | 4,256 pg/ml | 20,672 pg/ml | 27,824 pg/ml |
| [EpCAM] | | | | |
| Depleted | None measured | 4,160 pg/ml | 1,921 pg/ml | 3,016 pg/ml |
| [EpCAM] | | | | |
| Pellet | 204 pg/ml | 1022 pg/ml | 9,616 pg/ml | 14,032 pg/ml |
| [EpCAM] | | | | |
| Pellet | 851 μg/ml | 1022 μg/ml | 882 μg/ml | 1,178 μg/ml |
| total protein | | | | |
| Pellet EP/ | 0.65 | 0.24 | 0.46 | 0.5 |
| medium EP | | | | |
| g EpCAM/ | 4.8 × 10⁻⁶ | 2.4 × 10⁻⁵ | 1.92 × 10⁻⁴ | 2.8 × 10⁻⁴ |
| g total | | | | |
| protein | | | | |

If further analysis of the interior or contents of the exosome is desired (such as by antibodies against cytoplasmic proteins), the addition of formaldehyde followed by detergent will permeabilize the exosome membranes. Thus the detection antibodies may bind to interior exosome-associated antigens.

### Example 3

### Detection/Quantitation/Characterization of Exosomes by IHC

Exosome IHC will generally be carried out on an exosome block. Exosome blocks are prepared by isolating exosomes from a patient sample (e.g., plasma sample), fixing the exosome pellet (e.g., with formalin), and then embedding the fixed pellet in some medium (e.g., paraffin) that allows for convenient long-term storage. Qualitative and quantitative IHC analysis may then be performed on thin slices of the exosome blocks using antibodies against proteins of interest on the exterior or interior of the exosomes.

### Example 4

### Detection/Quantitation/Characterization of Exosomes by ECL ELISA

A variation of ELISA that may be used in the methods of the invention employs electrochemiluminescence (ECL). ECL ELISA is described in detail in, e.g., PCT Application Publication No. WO/1987/006706. Briefly, a sample is contacted with a detection antibody (e.g., anti-CD63) labeled with an electrochemiluminescent chemical moiety, the resulting sample is exposed to electrochemical energy, and the electromagnetic radiation emitted by the electrochemiluminescent chemical moiety is detected. The chief advantage of such an approach is the improved signal to noise ratio achieved using ECL. ECL can be extended beyond immunological assays to nucleic acid probe-based assays.

ECL ELISA was used to differentiate the plasma of cancer patients from the plasma of healthy controls. Plasma samples were obtained and diluted with 1% MSD Blocker A, without any initial enrichment for exosomes. The plasma samples used were as follows: 47 prostate cancer, 49 breast cancer, 48 colorectal cancer, 48 lung cancer, 50 ovarian cancer, and 70 healthy controls.

The Meso Scale Discovery® (“MSD”) platform was used in the following ECL ELISA experiments. MSD 96 well High Bind plates were incubated with a capture antibody (BD CD63—4 μg/ml) or protein (αVβ6 integrin—0.2 μg/ml) overnight. The plate was then washed three times with 3000 Wash Buffer (PBS 0.01% Tween). The plate was then passivated with 1% MSD Blocker A for 1 hour on an orbital shaker. The plate was then washed three times with 300 μl Wash Buffer. Diluted plasma samples were then allowed to incubate in the plate for 2 hours at room temperature on an orbital shaker. The plate was then washed three times with 300 μl Wash Buffer. 25 μl of the detection antibody was then loaded into each well and this was allowed to incubate for 1 hour at room temperature on an orbital shaker. The plate was then washed three times with 300 μl wash buffer.

The following antibodies were used:

TABLE 5

| Antibody Target | Vendor | Cat. # |
|---|---|---|
| CD63 | BD Biosciences | 556019 |
| Caudin-3 | R&D Systems | mab4620 |
| Claudin-4 | R&D Systems | mab4219 |
| CD36 | USBiological | c2388-04b |
| HLA-DR | eBioscience | 13-9956-82 |
| EpCAM | R&D Systems | 842008 |
| αVβ6 integrin | R&D Systems | 3817-AV |
| CD147 | AbCAM | AB666 |

In cases where the detection antibody had been directly labeled with the SULFO-TAG conjugate, 150 μl of 1× Read Buffer T (cat#R92TD-2) was added and the resulting sample was read on the MSD SECTOR Imager 6000. In cases where the antibody had not been not directly labeled the appropriate secondary antibody, e.g., SULFO-TAG Streptavidin, was used for detection. The secondary antibody was allowed to incubate for 30 minutes at room temperature on an orbital shaker. The plate was then washed three times with 300 μl Wash Buffer. 150 μl of 1× Read Buffer T (cat#R92TD-2) was added and the resulting sample was read on the MSD SECTOR Imager 6000.

The various capture-detection pairs differentiated cancers from controls as follows:

TABLE 6

| Disease | Marker Pair | p-value |
|---|---|---|
| Breast | αVβ6 integrin Capture -- CD63 Detect | 2.90E−13 |
| Breast | CD63 Capture -- CD36 Detect | 4.17E−12 |
| Breast | CD63 Capture -- CD147 Detect | 4.65E−08 |
| Breast | CD63 Capture -- Claudin-3 Detect | 2.65E−06 |
| Breast | CD63 Capture -- HLA-DR Detect | 6.00E−04 |
| Breast | αVβ6 integrin Capture -- CD147 Detect | 1.27E−03 |
| Breast | CD63 Capture -- EpCAM Detect | 2.99E−02 |
| Breast | CD63 Capture -- Claudin-4 Detect | 8.42E−02 |
| Colon/Rectal | αVβ6 integrin Capture -- CD63 Detect | 3.26E−22 |
| Colon/Rectal | CD63 Capture -- CD36 Detect | 2.93E−19 |
| Colon/Rectal | CD63 Capture -- CD147 Detect | 8.20E−14 |
| Colon/Rectal | αVβ6 integrin Capture -- CD147 Detect | 1.50E−09 |
| Colon/Rectal | CD63 Capture -- HLA-DR Detect | 2.66E−07 |
| Colon/Rectal | CD63 Capture -- Claudin-3 Detect | 2.71E−04 |
| Colon/Rectal | CD63 Capture -- EpCAM Detect | 3.19E−02 |
| Colon/Rectal | CD63 Capture -- Claudin-4 Detect | 3.87E−02 |
| Lung | CD63 Capture -- CD147 Detect | 3.20E−03 |
| Lung | αVβ6 integrin Capture -- CD63 Detect | 3.35E−03 |
| Lung | CD63 Capture -- CD36 Detect | 5.57E−03 |
| Lung | αVβ6 integrin Capture -- CD147 Detect | 1.02E−01 |
| Lung | CD63 Capture -- HLA-DR Detect | 1.47E−01 |
| Lung | CD63 Capture -- Claudin-3 Detect | 7.07E−01 |
| Lung | CD63 Capture -- Claudin-4 Detect | 8.08E−01 |
| Lung | CD63 Capture -- EpCAM Detect | 8.26E−01 |
| Ovarian | CD63 Capture -- Claudin-3 Detect | 2.98E−09 |
| Ovarian | CD63 Capture -- CD36 Detect | 2.00E−08 |
| Ovarian | αVβ6 integrin Capture -- CD63 Detect | 5.05E−08 |
| Ovarian | CD63 Capture -- HLA-DR Detect | 5.42E−07 |
| Ovarian | αVβ6 integrin Capture -- CD147 Detect | 3.44E−04 |
| Ovarian | CD63 Capture -- CD147 Detect | 4.01E−04 |
| Ovarian | CD63 Capture -- EpCAM Detect | 2.30E−02 |
| Ovarian | CD63 Capture -- Claudin-4 Detect | 8.12E−01 |
| Prostate | CD63 Capture -- CD36 Detect | 2.25E−07 |
| Prostate | CD63 Capture -- CD147 Detect | 7.56E−07 |
| Prostate | CD63 Capture -- Claudin-3 Detect | 7.38E−06 |
| Prostate | αVβ6 integrin Capture -- CD63 Detect | 1.89E−05 |
| Prostate | αVβ6 integrin Capture -- CD147 Detect | 1.99E−04 |
| Prostate | CD63 Capture -- Claudin-4 Detect | 8.11E−04 |
| Prostate | CD63 Capture -- HLA-DR Detect | 6.52E−03 |
| Prostate | CD63 Capture -- EpCAM Detect | 1.41E−02 |

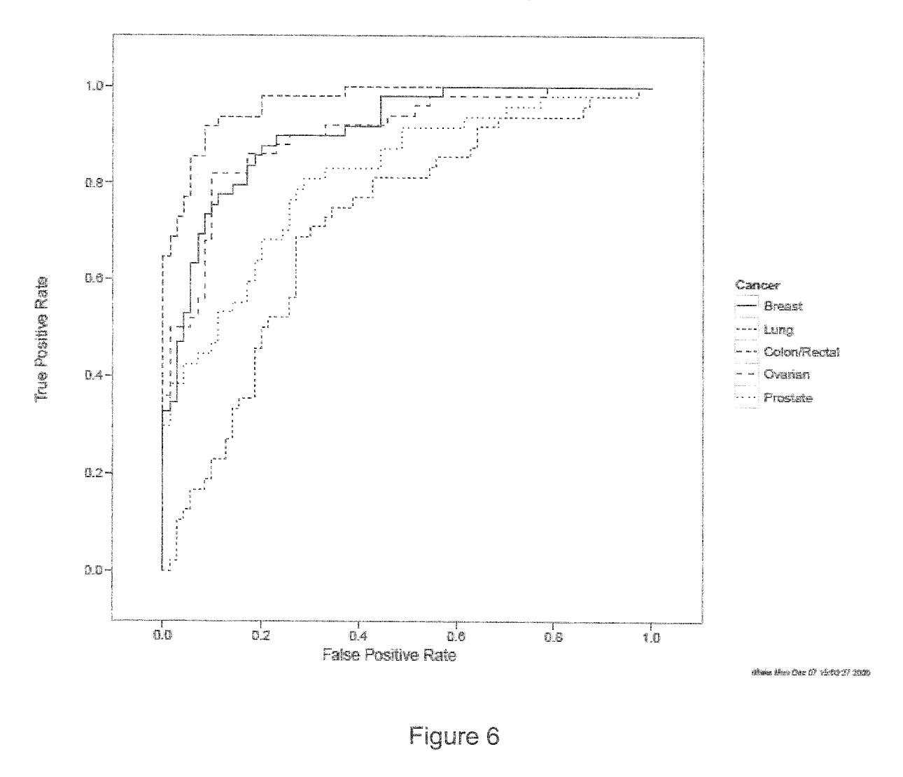

These correlations generally held up under multivariate analysis. See FIG. 6. Specifically, for CD36, Claudin-3 and Claudin-4:

TABLE 7

| Disease | p-value | AUC |
|---|---|---|
| Breast | 1.70E−12 | 0.88 |
| Lung | 4.00E−03 | 0.72 |
| Colon/Rectal | 3.70E−19 | 0.95 |
| Ovarian | 1.40E−08 | 0.81 |
| Prostate | 9.80E−08 | 0.81 |

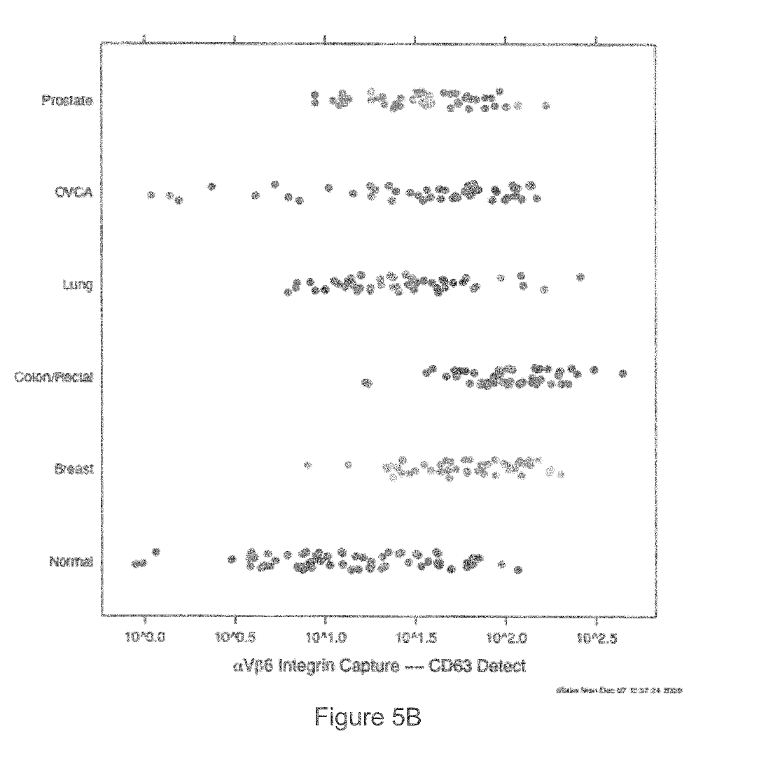

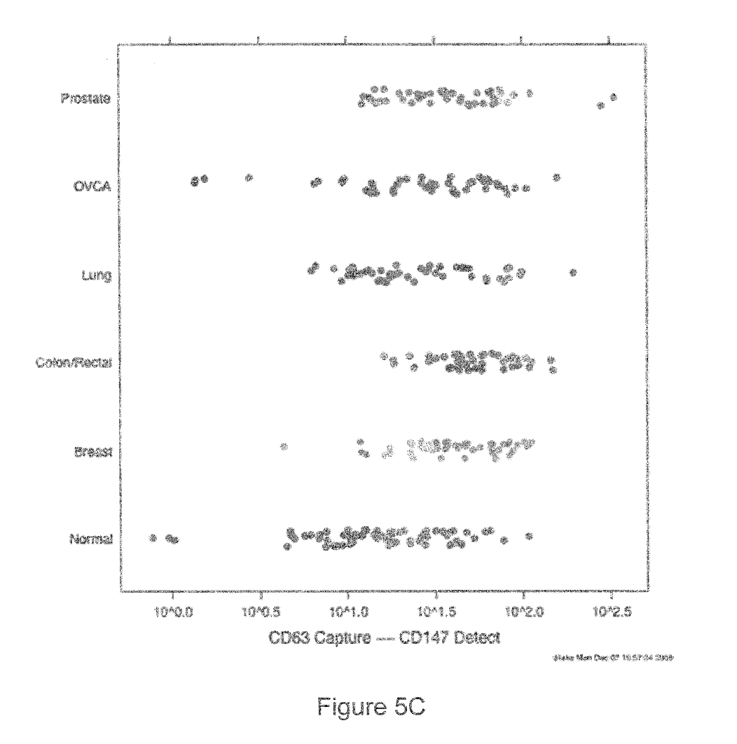

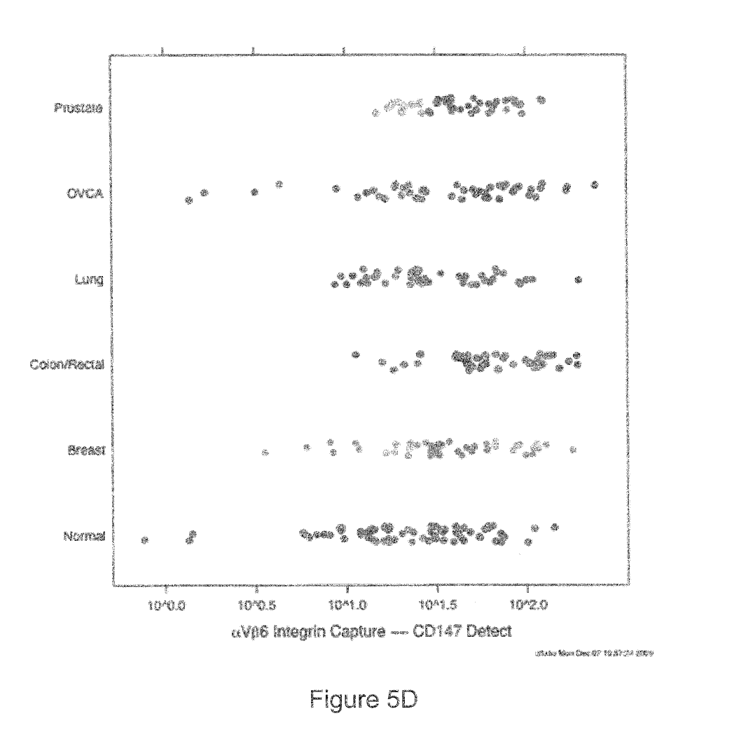

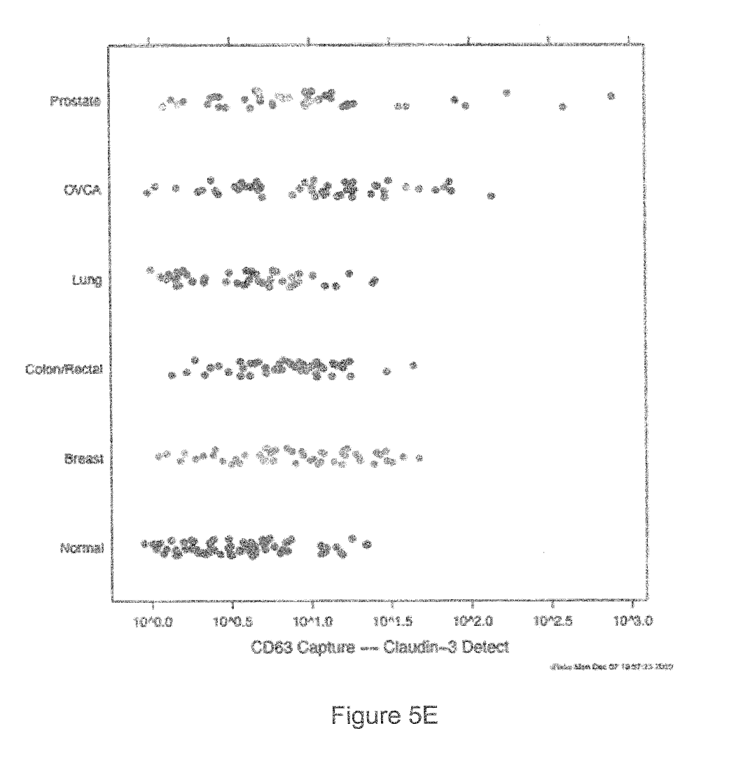

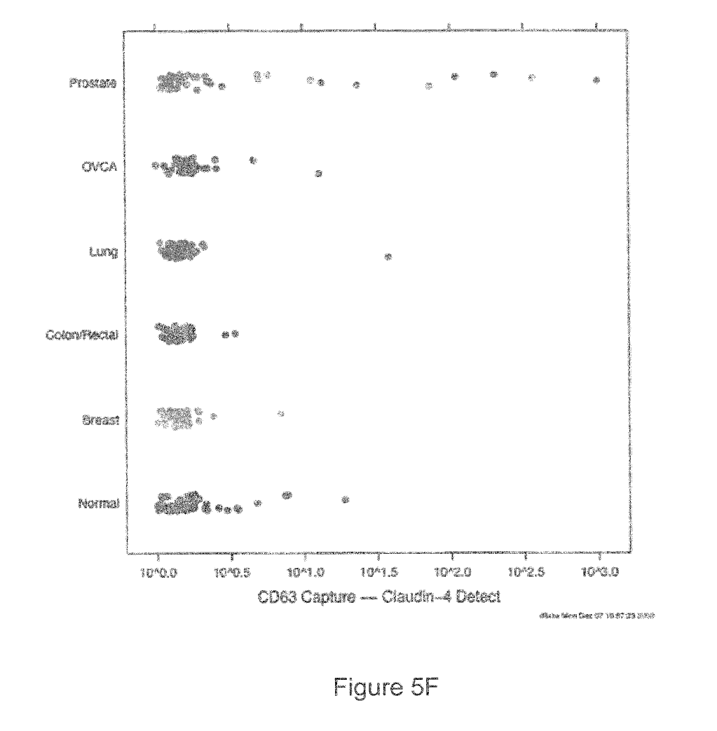

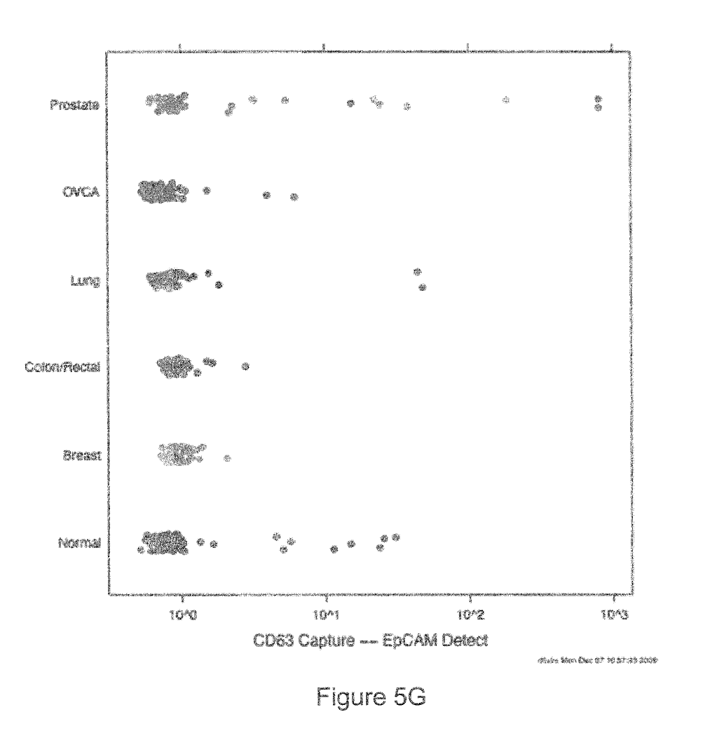

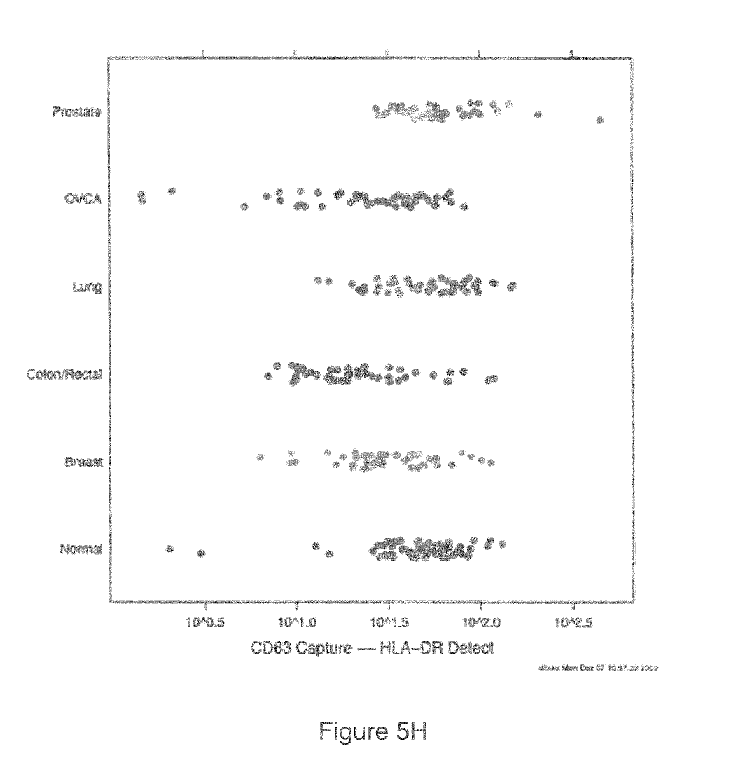

These results are summarized in FIG. 5. Thus the capture-detection pairs above can detect prostate, ovarian, lung, colorectal, and breast cancer in the plasma of patients.

### Example 5

### Detection/Quantitation/Characterization of Exosomes by ECL ELISA after Exosome Enrichment

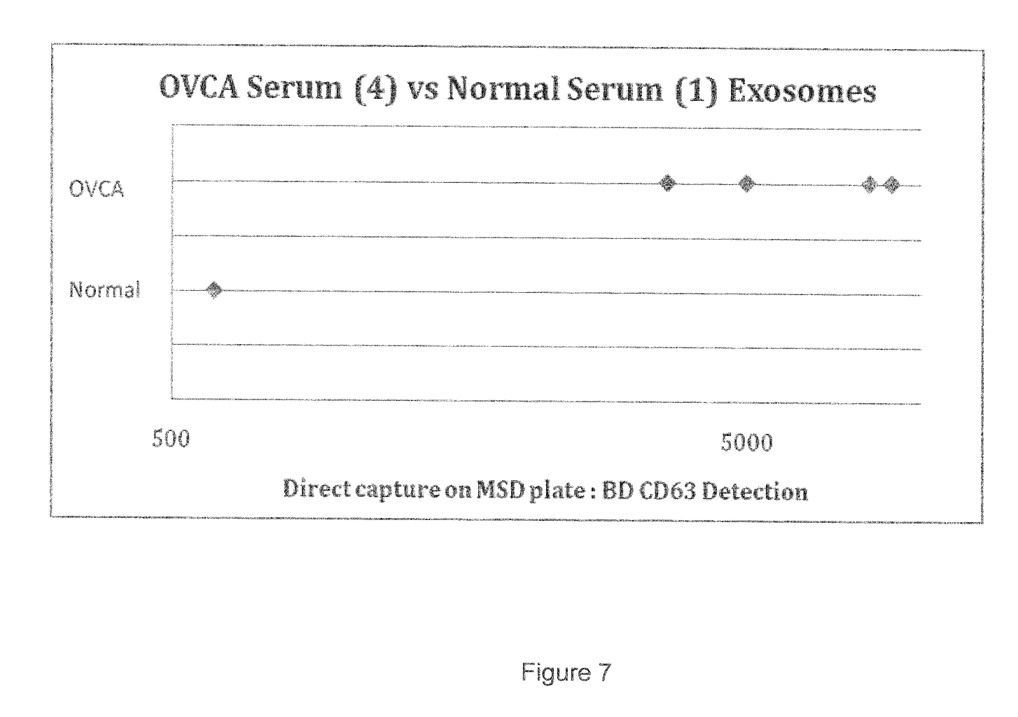

ECL ELISA was combined with differential centrifugation to separate ovarian cancer patients from controls. Differential centrifugation was performed as described in Example 1 above. ECL ELISA was performed on the resulting enriched samples essentially as described in Example 5 above, except that enriched exosome samples were directly captured on the MSD plate rather than on an MSD plate coated with a capture reagent. Specifically, 25 μl of each sample was the allowed to absorb onto an MSD 96 well High Bind Plate (Cat #L11XB-6). The plate was then passivated using MSD Blocker A (Cat #R93AA-1). The resulting plate was then incubated with SULFO-TAG labeled anti-CD63 antibody (CD63 BD Pharmingen Clone #H5C6) and read on a MSD SECTOR Imager 6000. The results are summarized in Table 8, below, and FIG. 7.

TABLE 8

| Sample Name | Signal |
|---|---|
| 256031 | 4970 |
| 256030 | 8872 |
| 256029 | 8127 |
| 256028 | 3624 |
| H4522 | 593 |

Although the foregoing invention has been described in some detail by way of illustration and example for purposes of clarity of understanding, it will be clear to those skilled in the art that certain changes and modifications may be practiced within the scope of the appended claims.

1-26. (canceled)

27. A method of detecting prostate cancer in a patient comprising determining the level of CD63 in a sample obtained from said patient; determining the level of at least one additional marker chosen from the group consisting of: ADAM10, αVβ6 integrin, Caveolin-1, CD147, CD36, CD63, CD81, Claudin-3, Claudin-4, Desmocollin-1, EGFR, EGFRvIII, EMP-2, EpCAM, ErbB2, GP1b, HLA-DR, Hsp70, Hsp90, MFG-E8, Rab13, Tissue Factor, CA-125, CEA, PSA, PSMA, PSAP, CK7, and CK20; and correlating an elevated level of CD63 and an elevated level of said additional marker in said sample to an increased likelihood of prostate cancer.

28. The method of claim 27, wherein said additional marker is EpCAM.

29. The method of claim 27, wherein said additional marker is PSMA.

30. The method of claim 29, further comprising determining the level of PSMA.

31. The method of claim 27, further comprising determining the level of PSA in a sample obtained from said patient and correlating an elevated level of PSA to the presence of prostate cancer.

32. The method of claim 31, wherein the level of PSA is determined by the quantification of PSA-bearing exosomes in said sample.

33. The method of claim 27, wherein said sample is chosen from the group consisting of blood sample, serum sample and plasma sample.

34. The method of claim 27, wherein said sample is enriched for exosomes.

35. The method of claim 34, wherein enriching said sample for exosomes comprises capturing exosomes on a solid surface.

36. The method of claim 27, wherein said level is determined by a technique chosen from the group consisting of: flow cytometry; ELISA; electrochemiluminescence ELISA; and IHC.

37. A method of diagnosing prostate cancer in a patient comprising identifying said patient as at risk of having prostate cancer; determining the level of CD63 in a sample obtained from said patient; determining the level of at least one additional marker chosen from the group consisting of: ADAM10, αVβ6 integrin, Caveolin-1, CD147, CD36, CD63, CD81, Claudin-3, Claudin-4, Desmocollin-1, EGFR, EGFRvIII, EMP-2, EpCAM, ErbB2, GP1b, HLA-DR, Hsp70, Hsp90, MFG-E8, Rab13, Tissue Factor, CA-125, CEA, PSA, PSMA, PSAP, CK7, and CK20; and correlating an elevated level of CD63 and an elevated level of said additional marker in said sample to an increased likelihood of prostate cancer.

38. The method of claim 37, wherein said additional marker is EpCAM.

39. The method of claim 37, wherein said additional marker is PSMA.

40. The method of claim 38, further comprising determining the level of PSMA.

41. The method of claim 37, further comprising determining the level of PSA in a sample obtained from said patient and correlating an elevated level of PSA to the presence of prostate cancer.

42. The method of claim 37, wherein said sample is chosen from the group consisting of blood sample, serum sample and plasma sample.

43. The method of claim 37, wherein identifying said patient as at risk of having prostate cancer comprises determining that said patient (a) had a suspicious digital rectal exam, (b) had a suspicious biopsy, or (c) has elevated levels of PSA.

44. The method of claim 37, wherein said status is determined by a technique chosen from the group consisting of: flow cytometry; ELISA; electrochemiluminescence ELISA; and IHC.

45. A kit comprising reagents for the detection of at least three of the markers chosen from the group consisting of ADAM10, αVβ6 integrin, Caveolin-1, CD147, CD36, CD63, CD81, Claudin-3, Claudin-4, Desmocollin-1, EGFR, EGFRvIII, EMP-2, EpCAM, ErbB2, GP1b, HLA-DR, Hsp70, Hsp90, MFG-E8, Rab13, Tissue Factor, CA-125, CEA, PSA, PSMA, PSAP, CK7, and CK20.

46. The kit of claim 45, wherein at least one of said reagents is attached to a solid surface.
